(19) United States
(12) Patent Application Publication
(10) Pub. No.: US 2024/0024707 A1
(43) Pub. Date: Jan. 25, 2024

(54) GENERIC MODULAR RESPIRATOR

(71) Applicant: AIMWELL HOLDING PTY LTD, New South Wales (AU)

(72) Inventor: Xiaoyi Fu, Macquarie Park (AU)

(73) Assignee: AIMWELL HOLDING PTY LTD, New South Wales (AU)

(21) Appl. No.: 18/256,393

(22) PCT Filed: Nov. 1, 2021
(86) PCT No.: PCT/AU2021/051280

§ 371 Date: Jun. 7, 2023

(30) Foreign Application Priority Data
Dec. 11, 2020 (AU) 2020904615

(51) Int. Cl. A62B 7/10; A62B 9/04; A62B 9/00; A62B 18/00; A62B 9/02; A62B 18/10; A62B 18/08; A62B 23/02

(52) U.S. Cl. CPC A62B 7/10; A62B 9/04; A62B 9/00; A62B 18/006; A62B 9/02; A62B 18/10; A62B 18/08; A62B 18/084; A62B 23/02

(57) ABSTRACT

A generic modular respirator comprising: a mask assembly having a first end and a second end; a replaceable main unit having a first housing with a first push-fit and twist-release connector to engage the first end of the mask, and a second housing with a second push-fit and twist-release connector to engage the second end of the mask; wherein the mask assembly and the main unit are adapted to perform breathing functions, such as breathing protection, breathing training, breathing measurement and diagnostics; such that the mask assembly is adapted to be quickly fitted to and released from the main unit by actuating the first and second push-fit and twist-release connectors.

### TECHNICAL FIELD

The present invention relates to a generic modular breathing apparatus or respirator, that can be configured or adapted for breathing protection, breathing training, breathing measurement and diagnostics.

### BACKGROUND

Air can contain particles which can be harmful or undesirable to the human body when inhaled. These particles may be breathed in by an individual without knowledge that the undesirable particles were even present. These particles may be for example in the form of bacteria, allergens, and viruses. Such particles can cause injury, create respiratory illnesses, cause general discomfort, and may even trigger allergic reactions. Different environments may have a higher level of exposure of harmful particles than others. While all individuals may inadvertently breathe in harmful particles, healthcare workers in high risk occupations with higher levels of exposure, and workers in polluted occupations with prolonged exposure to these harmful particles may be more susceptible to the negative effects the particles can cause.

In an effort to provide cleaner air for a person to breathe, conventional powered air purifying respirators, or PAPR, typically uses power to draw ambient air from the atmosphere through a filter element, and transfer it to the airways of the user via a conduit and mask. This ensures that the supply of air remains filtered or purified under all circumstances by maintaining a positive pressure inside the mask. However, the size of most PAPR systems is typically big and heavy. While known PAPR systems have filtering capability relocated to the hardhat or helmet, such as in US Published Patent Application No. U.S. Pat. No. 4,462,399, a possible disadvantage is that the device bulky and top-heavy for users' heads. Other known portable respirators such as in US Published Patent Application No. US20150314144 have an adjustable duct for air flow between the blower at the neck assembly and the mask assembly. Depending on the size of the user's head, the mask will be pressed upon the front of the user's face while the neck assembly will be pressed upon the back of the user's neck when in use. As no hardware and/or electronics is encapsulated by the duct, but rather encapsulated at the neck assembly, this renders the neck assembly lopsidedly heavy and may not be ergonomic. A possible disadvantage from the heavy neck assembly is that it can exert a lot of pressure down on the neck. Another possible disadvantage is that, when the neck assembly is too heavy or bulky, it can hinder head movements, in particular, during frequent left and right or up and down head movements. Another key disadvantage is that, for healthcare application, it is difficult to thoroughly clean or sterilize the airpath of the respirator, as significant airpath is inseparably connected with electronics and blower.

Air Purifying Respirator (APR) is another type of respirators that are similar to typical PAPRs in terms of reusability and elastomeric facial seal, with protection level sitting between typical PAPRs and dust or surgical mask. Compared with PAPRs, APRs are not powered, such that their protection and comfort level are not as good as PAPRs. However, being non-powered, they are smaller, lighter and cheaper than PAPRs; on the other hand, their facepieces are elastomeric which inherently provide better seal than dust and surgical masks. Therefore, they also play an important role in personal respiratory protection, except that most APRs are designed for industrial application only and not suitable to healthcare applications. The key shortcomings include most APRs having no exhalation filtration, having bulky inhalation filters fitted on the facepiece that affect field of view, lacking speech diaphragm that compromises communication, not upgradable to or interchangeable with PAPR, industrial looking and not aesthetically pleasing.

There is also a long felt need to provide a respirator for the public for general exercise to keep fit and/or to train for better lung capacity for recreational activities and/or extreme sports. Simulating and imposing breathing resistance on a respirator when training for an extreme environment with high altitude is desirable as a user does not require to always travel to the extreme environment to have a similar workout. A respirator for imposing breathing resistance is good for boosting lung capacity, strengthening breathing muscles as well as improving overall fitness of the user. While there are sports training masks available, most are not easily disassembled and/or difficult to clean and/or to replace parts. When wearing a training respirator, Other sports training masks are generally too bulky on the user's face and lack advanced functions or upgrade path, for example, other sports training masks only provide manually adjusted resistance and do not have electrically controlled or programmable resistance features for more advanced training. Another key issue is that among different sports training masks, each brand designs the resistance settings differently, thus it is impossible to relate these settings between different brands, and even with a fixed setting of a given mask, the real loading to the breathing muscles varies with breathing effort and simply can not be determined by the setting being used. Also, depending on the type of training required, there are no practical masks for measuring and recording the breathing performance during use, such as breathing rate, tidal volume, minute ventilation, amount of CO2 exhaled, etc., to gauge the efficiency of breathing, as well as level of fitness for the user.

Furthermore, there is an emerging trend and need to normalise one's breathing for vast majority of people of the modern era, as over 90% the world population more or less suffer from chronic hyperventilation (over-breathing) which if uncorrected, will lead to chronic health issues, such as asthma, heart disease, high blood pressure, even cancer. A wearable breathing measurement apparatus will provide a tool to help everyday person work towards a healthy breathing habit leading to a healthier life.

Currently, term ‘Respirator’ is often related to a device that provides respiratory protection. This is true when we refer to PAPRs and APRs, However, the definition of respirator goes far beyond respiratory protection. In general term, any devices that are used for passing air to breathing can be regarded as respirators. Thus, the respirators used for training breathing can be regarded as training respirators, and the respirators used for breathing measurement can be regarded as measurement respirators, and so on. So it is more appropriate to call respirators as functional respirators in this context to reflect the fact that respirators are not solely for protection purpose. Clearly, it can hardly find any respirator structures currently available that could be configured or adapted to perform as different functional respirators. As a result, the respirators for protection are totally different from those for breathing training. The key shortcomings include low interchangeable capability between these respirators to share common/modular components, low technology sharing and exchange between different functional respirators, and the situation hinders or slowdown innovation, technology breakthrough and applications of related breathing devices.

Attempts to make respirator modular have been seen in recent year. US Published Patent Application No. 43448804 have mating clips to releasably engage the mask and the Neck module. The key issue with this mating clip is the difficulty to operate without looking at it: it is not very easy to align with the receiving connector, because the ‘Oval’ like shape requires a precise orientation in order to insert into a mating part which is difficult when doing a blind connection during donning; the ‘Release’ button is also not intuitive to operate without practicing for some times; the mating clip does not allow free rotation between the connecting part, limiting the degree of freedom to make the connecting part closer to the sides of face following the facial profile which may vary from person to person.

Any discussion of the prior art throughout the specification should in no way be considered as an admission that such prior art is widely known or forms part of common general knowledge in the field.

### SUMMARY

### Problems to be Solved

It is an advantage of the present invention to provide a generic modular respirator that can be configured or adapted to be used for breathing protection, breathing training, breathing measurement and diagnostics.

It is an advantage of the present invention to provide a range of novel connecting means to facilitate the configuration or adaptation of the said generic modular respirator.

It is an advantage of the present invention to improve the ergonomic aspect of the invention by encapsulating different hardware at different parts of the respirator assembly such that the load is not too heavy on one side or end, when worn.

It is an advantage of the present invention to protect users from inhaling dust pathogens and or viruses such as COVID-19, when unexpected contact from an infected person occurs in a public space from exercising and/or social interaction.

It is an advantage of the present invention to provide a smaller, lighter, easier to assemble and wear, more comfortable, and aesthetically pleasing portable and modular respirator.

It is an advantage of the present invention to provide modular or interchangeable APRs and PAPRs to be effective and convenience to operate in a variety of applications for respiratory protection, either in industrial or healthcare settings, for sports or for use by everyday persons.

It is an advantage of the present invention to provide improvements for respirators for general breathing health, including but not limited to breathing measurement and diagnostics for healthcare, sports and for the general public.

It is an advantage of the present invention to provide a new and novel breathing training respirator with stepped resistance settings, and with electrically controlled or programmable resistance settings, and with breathing effort measurement to quantify the resistance loading that the respirator applied to the breathing muscles or as a quantified breathing efficiency indication for a given breath work.

It is an advantage of the present invention to provide a portable respirator that is easily connectable so the working respirator can be easily constructable and worn.

It is an advantage of the present invention to provide configurable means of the generic modular respirator to include bellow-clips, bellow-links, case-clips, clip-on connectors, bayonet or thread connectors, and push-fit and twist-release connectors.

It is an advantage to provide an interchangeable healthcare APR and healthcare PAPR that are also easy for sterilisation among many other features.

It is an advantage to provide an industrial PAPR with curved pleated prefilter, main filter with integrated airpath and bayonet connection, blower assembled in the bellows at the back of the neck, sealed airpath in the control unit, among many other features.

It is an advantage to provide a breathing measurement apparatus that is capable of measuring breath CO₂ and other breathing data for breathing health and breathing normalisation purposes.

It is an advantage to provide a basic training respirator with manual resistance settings.

It is an advantage to provide a training respirator with breathing measurement.

It is an advantage to provide a training respirator using breathing effort in the form of power of breathing and work of breathing to quantity the resistance load from the training respirator.

It is an advantage to provide a training respirator using breathing effort in the form of power of breathing and work of breathing to quantity the breathing efficiency during exercises.

It is an advantage to provide a training respirator with programmable breathing resistance control.

It is an advantage to provide a push-fit and twist-release connector with pairing air-passing stud.

It is an advantage to provide a bridge piece for the mask assembly that fits inlet valve, speech diaphragm, exhalation valve as well as stiffening the mask wall.

It is an advantage to provide a pressure sensing port arrangement for healthcare PAPR.

It is an advantage of the present invention to overcome or ameliorate at least one of the disadvantages of the prior art, or to provide a useful alternative.

### Means for Solving the Problem

A first aspect of the present invention may relate to a generic modular respirator comprising: a mask assembly having a first end and a second end; a replaceable main unit having a first housing with a first Push-fit & Twist-release connector to engage the first end of the mask with the first air-passing stud connector and a second housing with a second Push-fit & Twist-release connector to engage the second end of the mask with the second air-passing stud connector, wherein the mask assembly and the main unit are adapted to perform breathing functions, such as breathing protection, breathing training, breathing measurement and diagnostics; such that the mask assembly is adapted to be quickly fitted to and released from the main unit by actuating the first and second Push-fit & Twist-release connectors.

Preferably, the first housing and the second housing may each have a distal portion with a case-clip.

Preferably, the first housing and the second housing may each comprise a Clip-on module either at the bottom or at the top; preferably, the distal end of the first housing and the second housing may each comprises an add-on module.

Preferably, the first housing and the second housing may each comprise a middle portion with flexible concertina shaped walls. Preferably, the middle portion may be a connector assembly comprising a bellow and a clip. Preferably, the connector assembly may be adapted for connecting the proximal portion to the distal portion of the housings, such that the bellow allows the housings to arcuate when the neck abuts the middle portion.

Preferably, the first housing and the second housing may be connected by a neck assembly having a first end and a second end, wherein the first end of the neck assembly may be adapted to engage with a first distal end of the first housing with a bayonet or thread connector, and the second end of the neck assembly may be adapted to engage with a second distal end of the second housing with another bayonet or thread connector.

Preferably, the first Push-fit & Twist-release connector may be a first circular air-passing Push-fit socket with a first Twist-release ring, and wherein a second Push-fit & Twist-release connector may be a second circular air-passing Push-fit socket with a second Twist-release ring.

Preferably, the mask assembly may have a head strap assembly attachment means for securing a head strap assembly to the mask assembly. Preferably, the first housing and the second housing may each comprise a further head strap assembly attachment means or a clip for securing the first strap and the second strap of the head strap assembly to the first housing and the second housing respectively. Preferably, the mask assembly may have a speech diaphragm and an inlet valve. Preferably, there is a Bridge Piece to fit the speech diaphragm and the inlet valve inside the mask assembly.

Preferably, the main unit comprises at least one equipment adapted for use in at least one selected from a group of: assisting user respiration, respiratory protection, breathing training, and breathing measurement and diagnostics. More preferably, the portable respirator may further comprise a control unit with a power source, one or more sensors adapted for measuring breathing related physiological parameters. Preferably, the power source may be a rechargeable battery. Preferably, the rechargeable battery may be a lithium ion battery. Preferably, an equipment may be in electrical communication with another equipment. Preferably, the electrical communication may be by insulated electrical cables. More preferably, the equipment may be in wireless communication with another equipment and/or a remote terminal. Preferably, the mask may be adapted for covering oral and nasal passages. More preferably, the mask may further comprise an eye shield for covering the eyes of a wearer.

Preferably, in one preferred embodiment, the first housing may encapsulate a prefilter, a blower and a main filter. Preferably, the second housing may encapsulate a control unit with a power source and an exhaust filter. Preferably, the mask may be in fluid communication with the first housing and the second housing. Preferably, the blower may be adapted for pumping the filtered air from the first housing to the mask, wherein the first housing, the mask and the second housing may jointly form an air passage to pass therethrough. Preferably, the connection assembly of the first housing may comprise an airflow guard, wherein the blower may be received in the airflow guard. Preferably, the first distal portion of the first housing may have an air inlet, a prefilter and a blower; and preferably, a first proximal portion of the first housing may have a main filter and an air outlet. Preferably, the second proximal portion of the second housing may have an exhaust inlet, an exhaust outlet, exhalation valve and the exhaust filter; such that the exhalation valve and the exhaust filter may be positioned between the exhaust inlet and the exhaust outlet. Preferably, the first distal portion may have a prefilter slot adapted for receiving a prefilter frame, wherein the prefilter may be mountable to the prefilter frame; and preferably, a cross-sectional shape of the slot may be arcuate from an upper surface to a lower surface of the first distal end. Preferably, any one of the main filter and the exhaust filter may be adapted to be co-moulded or glued to a main filter frame or exhaust filter frame respectively, wherein the filter frame may have a rectanguloid profile. Preferably, the exhaust filter is a foam filter and may be washable. Preferably, the first distal end and the second distal end may each have a cable outlet for receiving insulated electrical cables such that the control unit may be in electrical communication with the blower. Preferably, the first connection assembly bellow-clip comprises a first bellow-link and a first case-clip, and wherein the second connection assembly bellow-clip comprises a second bellow-link and a second case-clip.

Preferably, the first bellow-link in the Bellow-Clip is flexible such that the first housing can arcuate outwardly away from the second housing. Preferably, the second bellow-link in the Bellow-Clip is flexible such that the second housing can arcuate outwardly away from the first housing. Preferably, a sound dampening material is mounted to the first connection assembly. Preferably, the mask further comprises a speech diaphragm assembly and an inlet valve. Preferably, the mask has an internal Bridge Piece that fits the speech diaphragm and the inlet valve.

Preferably, in another preferred embodiment, the first housing encapsulates a prefilter and a main filter; the second housing encapsulating an exhalation valve and an exhaust filter; the mask in fluid communication with the first housing and the second housing; wherein the first housing, the mask and the second housing jointly form an air passage to pass therethrough.

Preferably, in another preferred embodiment, a first distal end of the first housing may have an air flow dial adapted to adjust a size of an opening for air to enter the first housing, and for controlling inhalation resistance. Preferably, a second distal end of the second housing may have an exhaust membrane for exhaled air to exit the second housing. Preferably, the air flow dial may comprise a multi-step setting to set the opening into different predetermined sizes. Preferably, the exhaust membrane may have a pivot pin which may be off-center from the circular exhaust membrane thus the membrane works as a flap directing flow towards the far end of the pivot.

Preferably, the equipment may further comprise an electronic actuator to control the air flow dial based on the measured breathing related physiological parameters. Preferably, the electronic actuator may be one selected from a group of: a solenoid valve, and a blower for blowing against the direction of inhalation.

Preferably, the equipment may measure breathing effort to quantify the resistance loading to the breathing muscles or as an indication for breathing efficiency. Preferably, the equipment may also measure and record breathing data during use, such as breathe rate, tidal volume, minute ventilation.

Preferably, the first housing and the second housing may each contain a control unit, a battery, a flowmeter and an optional pressure sensor, wherein the battery in each housing can be charged independently from an external DC input, wherein the first housing can communicate with the second housing via wireless communication, wherein each housing can also communicate with a remote terminal via wireless communication, thus eliminating a need to have an electrical cable linking between the housings. Preferably, the second housing also contains a CO₂ sensor.

Preferably, in another preferred embodiment with the neck assembly, the first housing may encapsulate a battery and a desiccator; and preferably, the second housing may encapsulate a control unit and a breathing airway. Preferably, the mask may encapsulate a breath sampler. Preferably, a flexible concertina shaped neck assembly may encapsulate a pump and a CO₂ sensor. Preferably, the pump may be adapted for pumping sampled air from the breath sampler to the CO₂ sensor via the desiccator. Preferably, the control unit may be in electrical communication with the pump and the CO₂ sensor; wherein the control unit may measure and record the breathing related physiological data, and wherein the recorded data is wirelessly communicated from the control unit to a remote terminal unit. Preferably, the main unit may comprise an air inlet for air to enter the respirator, and an exhaust outlet for air to exit the respirator; and preferably, the main unit may further comprise a main filter for filtering air prior to entering the mask assembly. Preferably, the desiccator may be in fluid communication between the breath sampler, and the CO2 sensor. Preferably, the second housing comprises a single airway for air to enter to and exit from the respirator, as well as facilitating respiratory flow and pressure measurement.

Preferably, in another preferred embodiment with the neck assembly, the first housing may encapsulate a prefilter, and a main filter. Preferably, the second housing may encapsulate a control unit that comprises a main control PCB or controller with a pressure sensor, a sealed airpath and a battery. Preferably, the neck assembly may encapsulate a blower, wherein the blower may be adapted for pumping the filtered air from the first distal end of the first housing to the mask assembly via the second distal end of the second housing. Preferably, the controller may be in electrical communication with the blower; wherein the controller may measure the airflow in the main unit, and based on the airflow measurement, the controller may dynamically regulate the blowing strength of the blower such that a positive air flow to the mask assembly may be maintained. Preferably, the neck assembly may comprise a bellow connector engageable between a first bellowed portion and a second bellowed portion. Preferably, the first bellowed portion may be in connection with the first distal end of the first housing, and the second bellowed portion may be in connection with the second distal end of the second housing. Preferably, the bellow connector may be adapted to receive a blower therein. Preferably, the first housing has a first proximal end and a first distal end, wherein a first air inlet is positioned at the bottom of the first housing and a first air outlet is positioned at the first distal end; and wherein the second housing has a second proximal end and a second distal end; wherein a second air inlet is positioned at the second distal end, and wherein a second air outlet is positioned at the second proximal end, and wherein the sealed airpath connects the second air inlet and the second air outlet. Preferably, the mask has a first end and a second end, wherein a mask inlet may be positioned at the second end, wherein a mask outlet and/or exhaust valve may be positioned at the front of the mask. Preferably, the front of the mask assembly comprises an exhalation valve membrane or a speech diaphragm.

In the context of the present invention, the words “comprise”, “comprising” and the like are to be construed in their inclusive, as opposed to their exclusive, sense, that is in the sense of “including, but not limited to”.

The invention is to be interpreted with reference to at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1a illustrates a generic modular respirator with main unit having a first housing and a second housing in connection to the mask assembly. This generic modular respirator may have a flexible middle portion or bellow-link in each housing.

FIG. 1b shows the first and second housing without a middle portion.

FIG. 2 illustrates a main unit of a generic modular respirator with an insulated electrical cable in connection between the first distal end of the first housing and the second distal end of the second housing.

FIG. 3 illustrates another embodiment of main unit of a generic modular respirator with a bellowed neck assembly in connection between the first distal end of the first housing and the second distal end of the second housing.

FIG. 4 illustrates another embodiment with a neck assembly of a generic modular respirator in which the first housing and the second housing are elongate members without the flexible middle portion or bellow-link in each housing, where the neck assembly connects to the distal end of the first housing with a bayonet or thread connector, and the neck assembly connects to the distal end of the second housing with another bayonet or thread connector.

FIG. 5 illustrates another embodiment with a neck assembly of a generic modular respirator in which the first housing and the second housing are elongate members with the flexible middle portion or bellow-link in each housing.

FIG. 6 illustrates FIG. 1 with an insulated electrical cable of a generic modular respirator in connection between the first distal end of the first housing and the second distal end of the second housing.

FIG. 7 illustrates an exploded view of a Push-fit & Twist-release connector used for connecting the ends of the main unit to the ends of the mask assembly.

FIG. 8 illustrates a side view of the connection assembly Bellow-Clip comprising a bellow-link and a case-clip for connecting the proximal portion with the distal portion of a housing together.

FIG. 9 illustrates a front view of FIG. 8.

FIG. 10 illustrates an exploded view of a head strap assembly embodiment.

FIG. 11 illustrates a dust cover covering any embodiments of the respirator.

FIG. 12 illustrates a perspective view of an assembled healthcare respirator.

FIG. 13 illustrates FIG. 12 without the head strap assembly with the mask assembly disengaged with the main unit.

FIG. 14 illustrates FIG. 12 without the head strap assembly and also without the cable cover.

FIG. 15 illustrates FIG. 12 without the head strap assembly with the proximal portion disengaged with the distal portion showing the Bellow-Clip connector.

FIG. 16 illustrates FIG. 15 with the mask assembly connected to the proximal portions while showing the opening of the Bellow-Clip connector adapted for receiving the blower therethrough.

FIG. 17 illustrates another perspective view of FIG. 12 showing the air inlet and where a prefilter frame can be slotted at the first distal end of the first housing.

FIG. 18 illustrates a side view of the FIG. 17.

FIG. 19 illustrates a cross-sectional view of FIG. 12, showing the airpath and components.

FIG. 20 illustrates a generic mask with a speech diaphragm with a front cover.

FIG. 21 illustrates an exploded view of the blower unit encapsulated by the distal portion of the first housing.

FIG. 22 illustrates the main filter frame with the main filter (left) and an exhaust filter frame with the exhaust filter (right).

FIG. 23 illustrates another preferred embodiment of an assembled healthcare respirator without electronics.

FIG. 24 illustrates another perspective view of FIG. 23.

FIG. 25 illustrates a perspective view of the second distal portion with a case-clip disengaged from the second housing of the embodiment illustrated in FIG. 23.

FIG. 26 illustrates a perspective view of the first distal portion with a case-clip disengaged from the first housing of the embodiment illustrated in FIG. 23.

FIG. 27a illustrates a perspective view of a breathing training respirator with a head strap assembly.

FIG. 27b illustrates a perspective view of a breathing training respirator with a head strap assembly and with the first housing and the second housing detached.

FIG. 28 illustrates another perspective view of FIG. 27a.

FIG. 29 illustrates a cross-sectional view of FIG. 27a.

FIG. 30 illustrates an assembled airflow dial.

FIG. 31 illustrates a second plate of the airflow dial of FIG. 30

FIG. 32 illustrates a back view of the first plate of the airflow dial of FIG. 30.

FIG. 33 illustrates a front view of the first plate of the airflow dial of FIG. 32.

FIG. 34 illustrates an assembled exhaust valve with the exhaust membrane.

FIG. 35 illustrates an exhaust membrane frame of FIG. 34.

FIG. 36 illustrates an off-centre pivoted exhaust membrane of FIG. 34.

FIG. 37 illustrates another preferred embodiment of a breathing training respirator with a head strap assembly showing elongated housings with Clip-on modules for encapsulating electronics for breathing measurement.

FIG. 38 illustrates a perspective view of FIG. 37.

FIG. 39 illustrates a back view of FIG. 37.

FIG. 40 illustrates another preferred embodiment adapted for use in breathing measurement.

FIG. 41 illustrates another preferred embodiment adapted for protection use in occupations with fine particulates.

FIG. 42 illustrates FIG. 41 with the mask disengaged with the first and second housing via the Push-fit & Twist-release connectors while the first housing is also disengaged with the first end of the neck assembly via a bayonet connector.

FIG. 43 illustrates a cross-sectional view of FIG. 41 showing the airpath.

FIG. 44 illustrates a bottom view of FIG. 41 showing the air inlet.

FIG. 45 illustrates a back view of FIG. 41 with a head strap assembly showing the hook and loop of the strap attachment engaging with the neckpad.

FIG. 46 illustrates a perspective view of FIG. 45.

FIG. 47 illustrates an exploded view of the mask assembly used in FIG. 41.

FIG. 48 illustrates an exploded view of the second housing and the neck assembly.

FIG. 49 illustrates an exploded view of the sealed airpath in the second housing.

FIG. 50 illustrates an exploded view of the first housing

FIG. 51 illustrates a main filter frame with an integrated airpath and a bayonet connector for connecting to the first end of the neck assembly.

FIG. 52 illustrates FIG. 51 with a main filter glued or co-moulded with the main filter frame with the integrated airpath.

FIG. 53 illustrates a lower surface of the first housing being disengaged showing the locking mechanism.

FIG. 54 illustrates a tether used for connecting a shell half with another shell half of the first housing when the shell halves are apart, and the main filter fitted in one of the shell halves.

FIG. 55 illustrates a curved pleated pre-filter for respirator 500.

FIG. 56a illustrates a rear view of the generic mask with a Bridge Piece to fit a speech diaphragm and an inlet valve inside the mask assembly.

FIG. 56b illustrates a section view of the generic mask of FIG. 56a.

FIG. 56c illustrates a front view of the Bridge Piece with cover 168.

FIG. 56d illustrates a rear view of the Bridge Piece.

FIG. 57 illustrates a rectangular exhalation valve membrane with a long pivot for respirator 200.

FIG. 58 illustrates another embodiment of a generic respirator with a detachably connected module at the bottom of the first housing and the second housing.

FIGS. 59a and 59b illustrates a Clip-on module that can be detachable from the bottom of the first housing and the second housing, and an add-on module that is in fixed connection with the distal portion of the first housing and the second housing.

FIGS. 59c and 59d illustrates an add-on module with push-fit and twist-release connection with the distal portion of the first housing and the second housing.

FIG. 60 illustrates a sensing port 185a and 185b between the second proximal portion 123 and second distal portion 124 of the second housing of respirator 200.

### DESCRIPTION OF THE INVENTION

Preferred embodiments of the invention will now be described with reference to the accompanying drawings and non-limiting examples.

The present invention relates to a generic modular respirator having quick replaceable parts that can be easily dissembled, assembled, configured and adapted by manipulating one of more of the uniquely designed connectors, including but not limited to Push-fit & Twist-release, Bellow-Clip, Below-link, case-clip, Bayonet or thread, Clip-on connectors into a specialised respirator dedicated for a specific purpose such as, breathing protection, breathing training, breathing measurement and diagnostics. Previously, respirators are designed for a single purpose or specialty and not suitable for another purpose. Specifically, breathing protection, breathing training, breathing measurement and diagnostics are common breathing needs by a large proportion of world population, however, a user may need multiple independent respirators for each of the needs. This increases the costs and waste a lot of resources for maintenance. There would also be low interchangeable capability between these respirators to share common/modular components, low technology sharing and exchange between different functional respirators, and the situation hinders or slowdown innovation, technology breakthrough and eventual applications of the related breathing devices. The present invention aims to provide a generic modular respirator to address the above mentioned issues.

Generic Modular Respirator

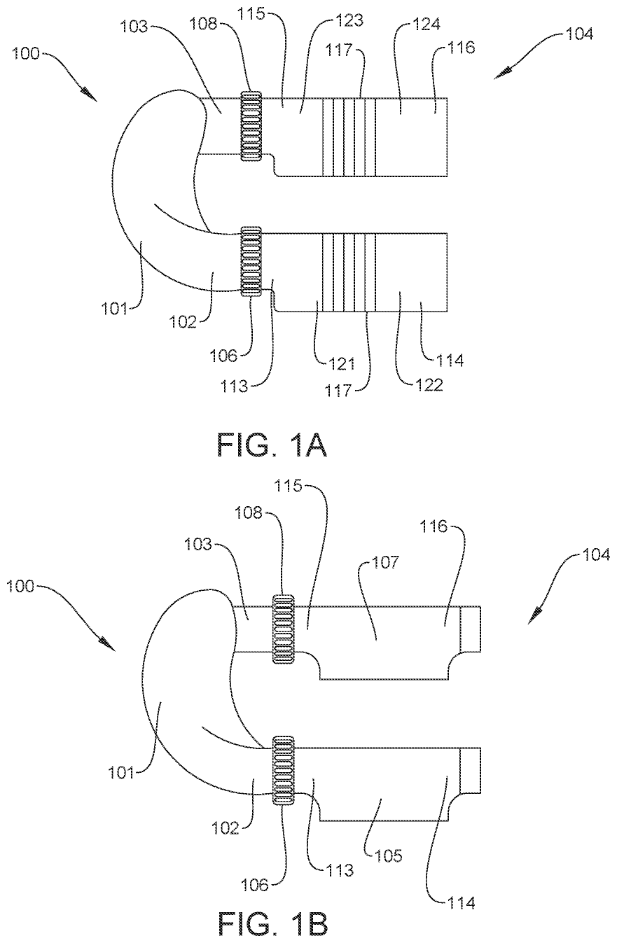

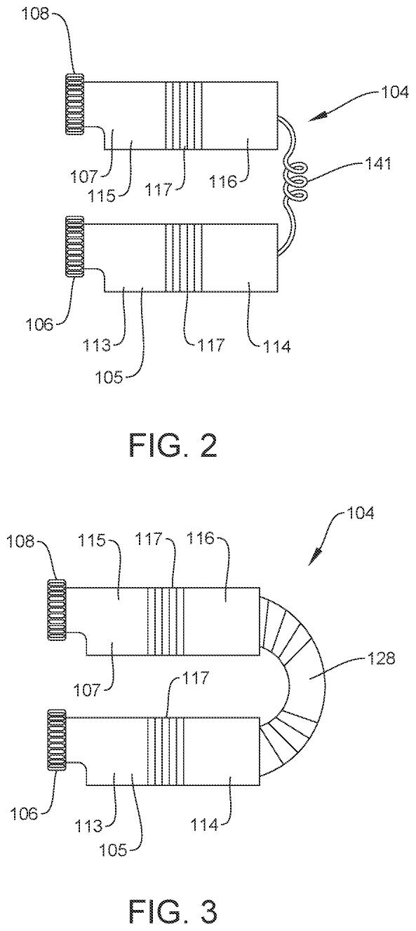

In an embodiment of the present invention, as illustrated in FIGS. 1 to 6, and FIG. 58, shows a portable respirator or a generic respirator 100 with a mask assembly 101 having a first end 102 and a second end 103; a replaceable main unit 104 having a first housing 105 with a first Push-fit & Twist-release connector 106 to engage the first end of the mask 102 and a second housing 107 with a second Push-fit & Twist-release connector 108 to engage the second end of the mask 103. Each end of the mask 102 & 103 has a pairing air-passing stud connector to connect with the Push-fit & Twist-release connector. In one preferred embodiment, shown in FIG. 1a, 1b, 58, there may be no electrical cable or neck assembly connected between the first housing 105 and the second housing 107. In another preferred embodiment, one of the first housing 105 and the second housing 107 is adapted to perform breathing functions. In another preferred embodiment, as shown in FIGS. 3 to 5, the main unit 104 may further comprise a neck assembly 128. The neck assembly 128 may have a first end 128a and a second end 128b. The first end 128a of the neck assembly may be adapted to engage with a first distal end of the first housing 105 or to the first distal portion of the first housing either with a fixed connection or with a releasable bayonet or thread connector 164. Similarly, the second end 128b of the neck assembly may be adapted to engage with a second distal end of the second housing 107 or to the second distal portion of the second housing either with a fixed connection or with a releasable bayonet or thread connector 164. The neck assembly may provide a channel for fluid communication and/or for electrical communication between the first housing/the first distal portion of the first housing, and the second housing/the second distal portion of the second housing, which is shown in FIG. 2-6. The main unit 104 may be adapted to control airflow in the first housing 105, the second housing 107, the neck assembly 128 and the mask assembly 101.

The first Push-fit & Twist-release connector 106 or the second Push-fit & Twist-release connector 108 used for the respirators is illustrated in FIG. 7. The first Push-fit & Twist-release connector 106 or the second Push-fit & Twist-release connector 108 may each have a circular air-passing Push-fit socket 109 with a control ring or a gripping ring 110 and a latching spring. The Push-fit socket has a lumen providing an air path for air to pass through. It also has an internal sealing sleeve to ensure leak-tight seal with the pairing air-passing stud connector in the mask assembly 101. The retaining spring has a first arm 112 latched on the griping ring 110 and the second arm 111 latching on the socket 109. In one embodiment, the first Push-fit & Twist-release connector 106 is releasable by rotation or a twist. Similarly, the second Push-fit & Twist-release connector 108 is releasable by rotation or a twist. In a locked position when the control ring is relaxed, spring arms 111/112 clamp to a locking slot in the air-passing stud of the mask assembly thus connecting with the mask assembly; when the control ring is rotated or twisted, spring arms 111/112 expands to be outside of the locking slot in the air-passing stud thus releasing the mask assembly. Being circular, it makes blind connection with the pairing air-passing stud very easy, it also allows free rotation between the socket and the pairing air-passing stud, thus allowing the connecting main unit more closely positioned against the contour of the face for better wearability. The circular Push-fit & Twist-release connectors 106/108 can provide an easier and better secured connection than non-circular insertion clip button type connectors. The mask assembly 101 may be adapted to be released from the main unit 104 by twisting or rotating the first Push-fit & Twist-release connector 106 and the second Push-fit & Twist-release connector 108. A wearer may twist or rotate the control ring 110 of the first Push-fit & Twist-release connector 106 and the second Push-fit & Twist-release connector 108 to push the first arcuate arm 111 away from the second arcuate arm 112 so that the pairing air-passing stud in the first end 102 and the second end 103 of the mask assembly 101 are no longer retained by the arcuate arms 111, 112 of the of the retaining ring. The mask assembly may then be released from the circular Push-fit connect sockets 109 respectively.

In another embodiment, the first Push-fit & Twist-release connector 106 or the second Push-fit & Twist-release connector 108 may have other kinds of retaining structure, such as a turn slot, circlip, clamps, insertion hooks, for releasably holding the pairing air-passing studs in the first end 102 and the second end 103 of the mask assembly 101.

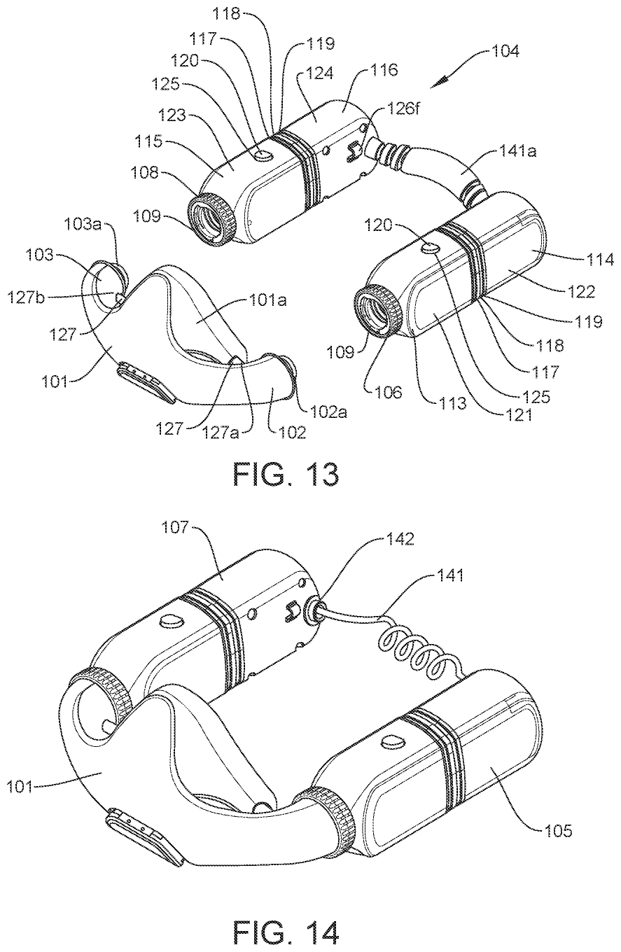

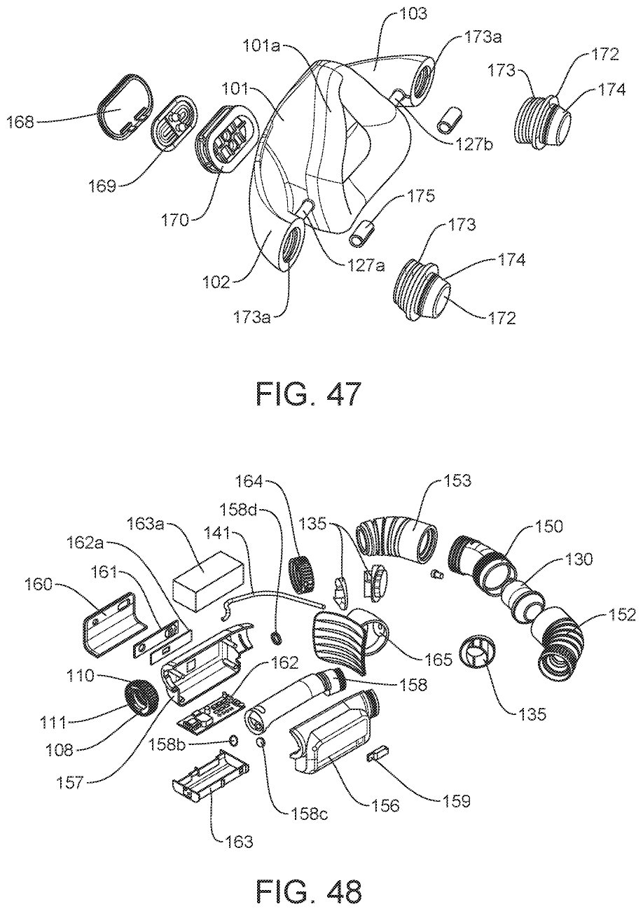

As shown in FIGS. 13 and 20, the mask assembly 101 may have a mask 101a for covering at least the oral and nasal passage of a user. The first end 102 and the second end 103 of the mask assembly 101 may each have an opening or an inlet fitted with a pairing air-passing stud connector which may be engageable with the circular Push-fit socket 109 of the first or second Push-fit & Twist-release connectors 106/108 respectively. As illustrated in the mask assembly 101 shown in an embodiment in FIG. 47, there may be a pairing air-passing stud 172 with a lumen extending from one end to the other end of the stud. The stud 172 may be positioned between the first end of the mask assembly 102 and the first Push-fit & Twist-release connector 106; and similarly, there may be another pairing air-passing stud 172 positioned between the second end of the mask assembly 103 and the second Push-fit & Twist-release connector 108. One end of the air-passing studs 172 may each have circular ribs for tightly engaging with the threads 173a in the opening of the first end and the second end of the mask assembly respectively. While the other end of the air-passing stud may have a circular slot and chamfered protrusion 174 that may engage and connect with the circular Push-fit connect socket 109 forming a sealed air passage between the first housing 105, the mask assembly 101 and the second housing 107, when in use. There may be an exhalation valve 169 and frame 170 at the front of the mask with a front mask cover 168

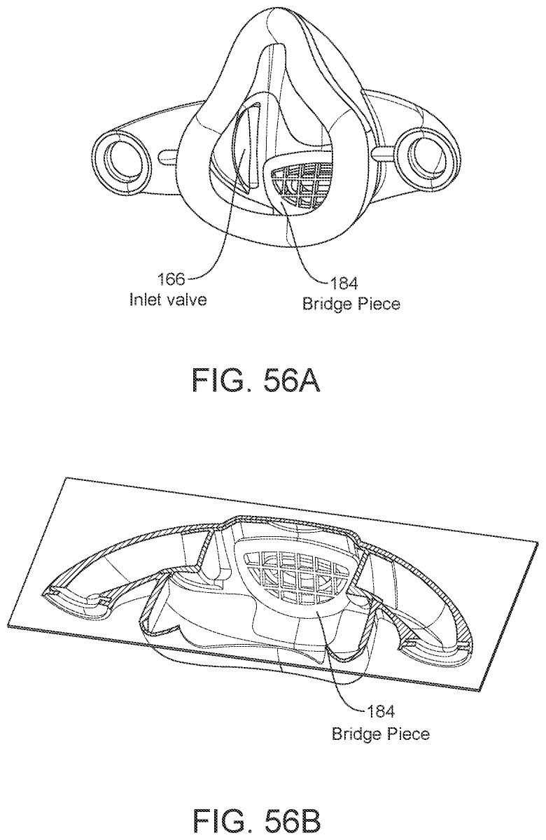

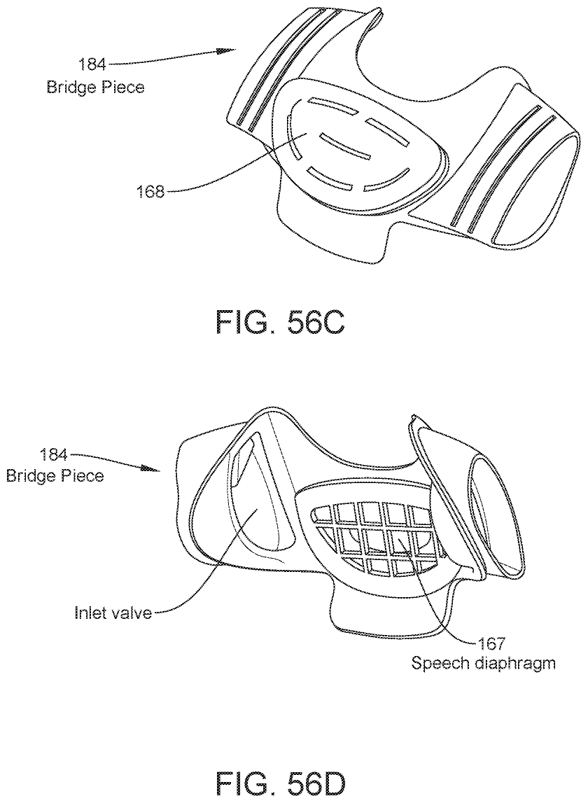

In one embodiment, as shown in FIG. 56, the mask assembly 101 may have an internal Bridge Piece 184 that fits an inlet valve 166 and a speech diaphragm 167. The Bridge Piece also stiffens the middle section of the mask to avoid collapsing under negative pressure during high exertion breathing. In another embodiment, 2 inlet valves may be fitted, and an exhalation valve may replace the speech diaphragm. The multi-function Bridge Piece helps simplify manufacturing and make the mask more compact and lighter. In another embodiment, there may be two inlet valves when adding another one at the other end of the Bridge Piece, and an exhalation valve may be fitted replacing the speech diaphragm.

In one embodiment, the mask 101a may be customised to fit upon the face of a user. This customised mask 101a can be manufactured by 3D scanning the face of the user and then 3D printing a mask that conform the contour of the user's face. This mask 101a can be made with a material that can be subjected to sterilisation. In this way, the mask 101a can be used for different purposes by replacing or swapping the main unit 104. In another embodiment, the mask 101a is a full face mask, covering the eyes as well.

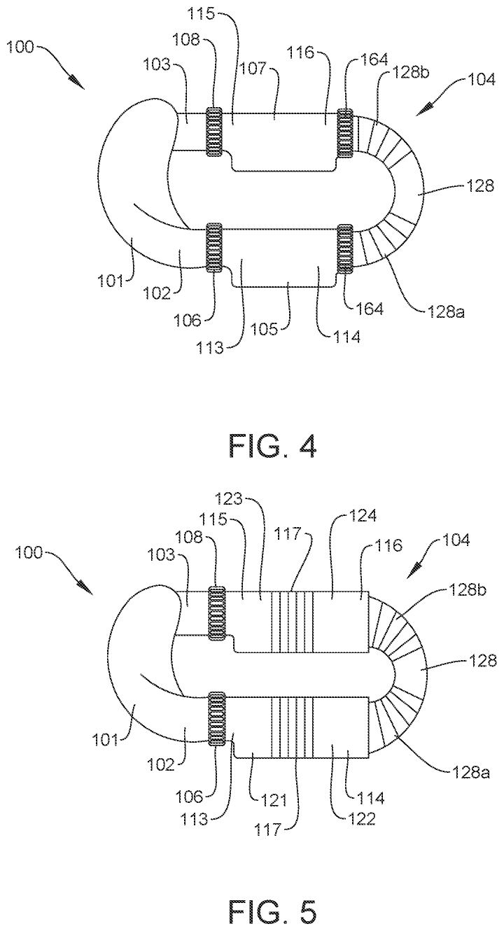

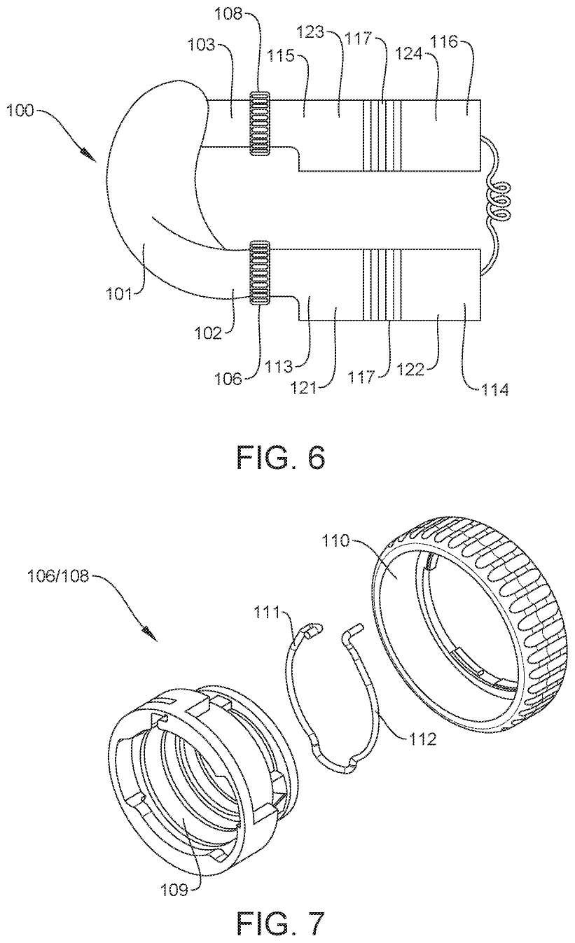

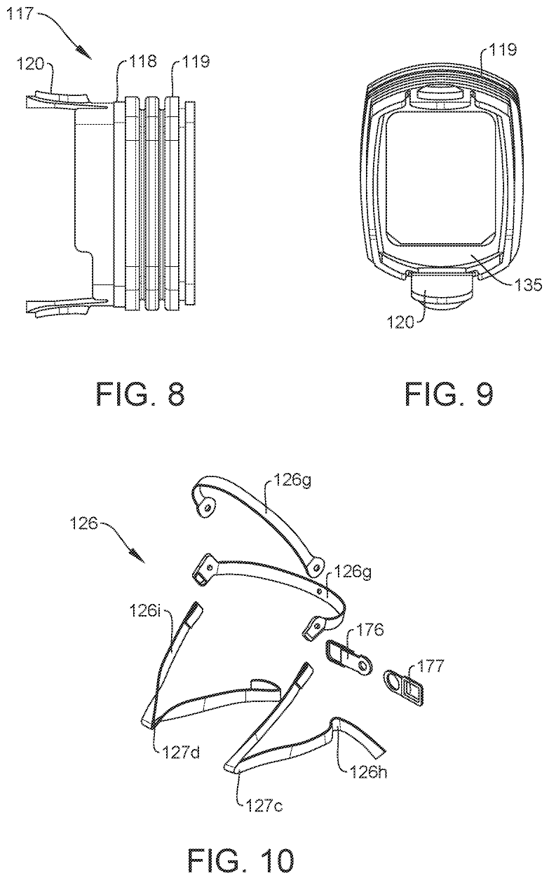

As shown in FIG. 1, FIGS. 4 to 6, the generic respirator 100 may have a first housing 105 and a second housing 107 that may each be elongate and modular. The first housing 105 may have a first proximal end 113 and a first distal end 114, and similarly, the second housing 107 may have a second proximal end 115 and a second distal end 116. In one preferred embodiment, as shown in FIGS. 1b and 4, the first housing and the second housing may each have a casing without a middle connection portion. In another preferred embodiment, as shown in FIG. 1a, FIGS. 5 and 6, the first housing 105 may have a first proximal portion 121 and a first distal portion 122, and similarly, the second housing 107 may have a second proximal portion 123 and a second distal portion 124. The first housing 105 and the second housing 107 may each comprise a middle portion 117 with flexible concertina shaped walls or a middle bellow portion. As shown in FIGS. 8 and 9, the middle portion 117 may be a connector assembly Bellow-clip 118 comprising a bellow-link 119 and a case-clip 120 that may be adapted for connecting the proximal portion and the distal portion of the first housing or the second housing together. The distal portion of the first housing or the second housing may be directly connected to the Bellow-clip 118, in which the proximal portion of the first housing or the second housing may have a clip engagement means 125 as shown in FIG. 13 for coupling the case-clip 120 (bottom clip not shown). Advantages of having detachable proximal portion or distal portion are that the equipment and/or electronics can be easily accessed by the wearer for servicing, replacing filters, replacing equipment and/or sensors, and ease of cleaning, for example. The other advantage having detachable proximal portion or distal portion with the Bellow-clip is that in case the housing needs to be long enough to fit internal components, the resulting distal portion of the housing can be positioned closer to the contour of the head/neck thanks to the flexible bellow, thus improving wearability. Yet another important advantage is that the Bellow-clip can achieve modularity for different functional respirators, where the proximal portion of the housing can be a common module, and the distal portion of the housing connected by the Bellow-link can have different functionalities for different functional respirators. Hence, the mask assembly 101 and the proximal portion of the first and second housing may become commonly shared portions among seemingly different functional respirators. The Bellow-Clip arrangement also allows servicing the proximal portion of the housing and the distal portion of the housing differently. Preferably, the bellow-link 119 has a small number of concertina shaped walls. Preferably, between two to four concertina shaped walls to permit some flexibility for the first housing or second housing to arcuate around the wearer's neck when the wearer's neck is abutting or pressing against the middle portion of the housings, when in the portable respirator is in use. Desirably, three concertina shaped walls may be chosen.

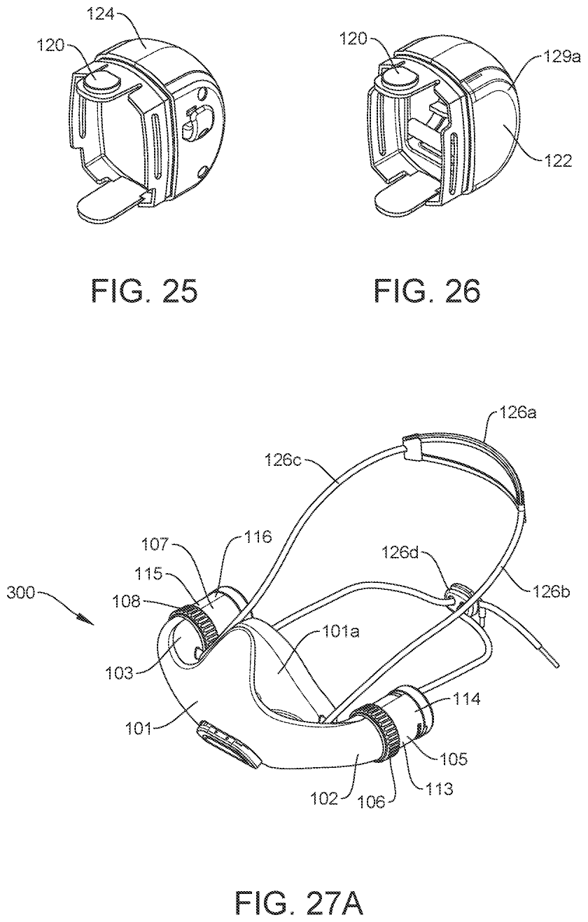

In another preferred embodiment, the middle portion 117 may have a case-clip 120 only as shown in FIG. 25 and FIG. 26. Such a variation is more suitable for a shorter first and second housing.

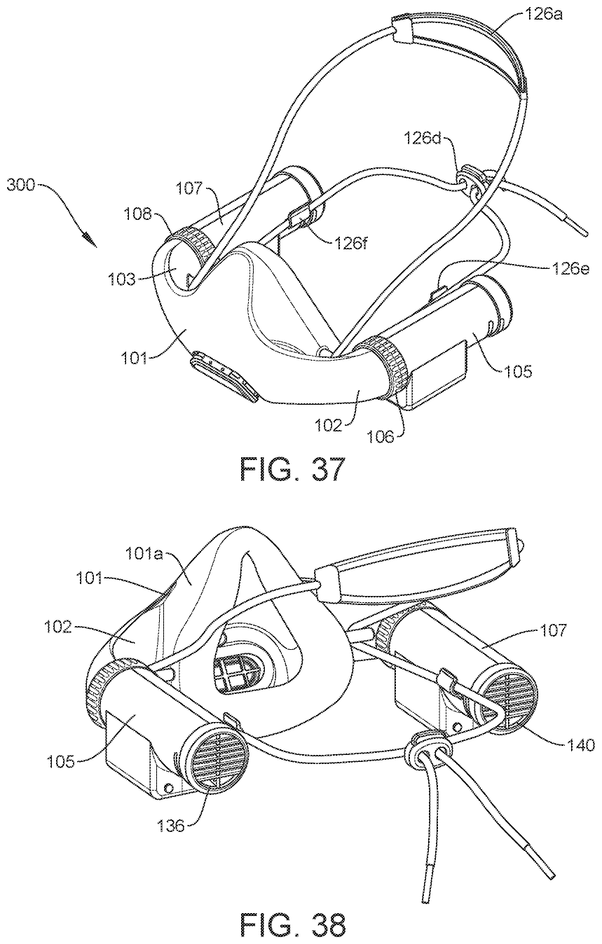

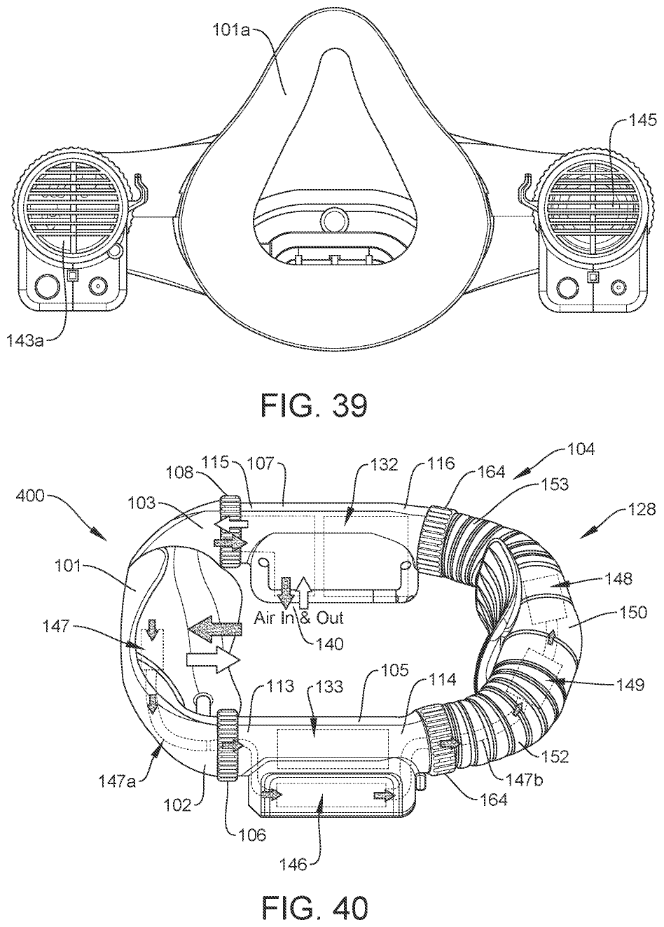

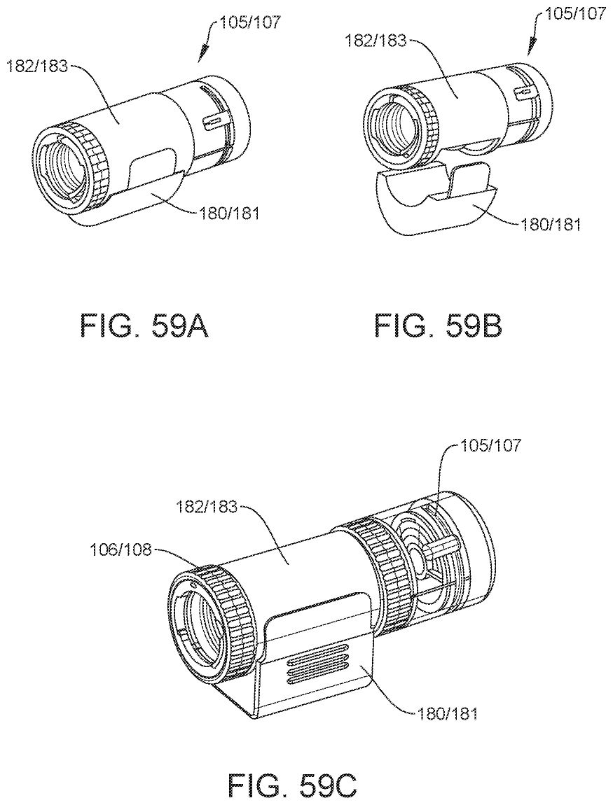

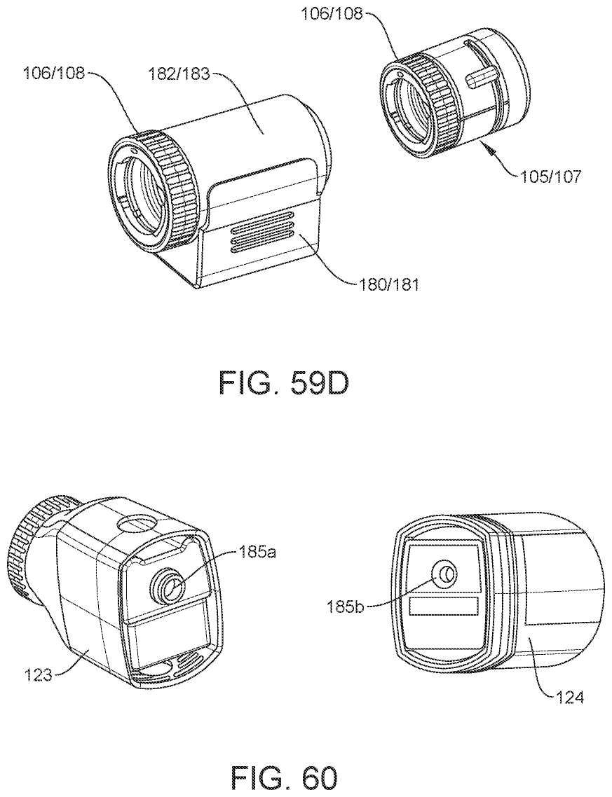

In another preferred embodiment, as shown in FIG. 37, 38, 39, FIG. 58, the first housing 105 may have a module 180 releasably attached at the bottom of the housing. Similarly, the second housing 107 may have a module 181 releasably attached at the bottom of the housing. As shown in FIG. 59a, 59b, module 180/181 each may have a Clip-on mechanism to releasably couple onto the bottom of the housing 105/107 respectively. The distal end of the first housing 105 and second housing 107 each may have an add-on module in fixed connection with the housing. In another preferred embodiment, as shown in FIGS. 59c and 59d, at the distal end of module 182/183 each may have an add-on module with push-fit and twist-release connection to releasably couple onto the distal end of 182/183 respectively.

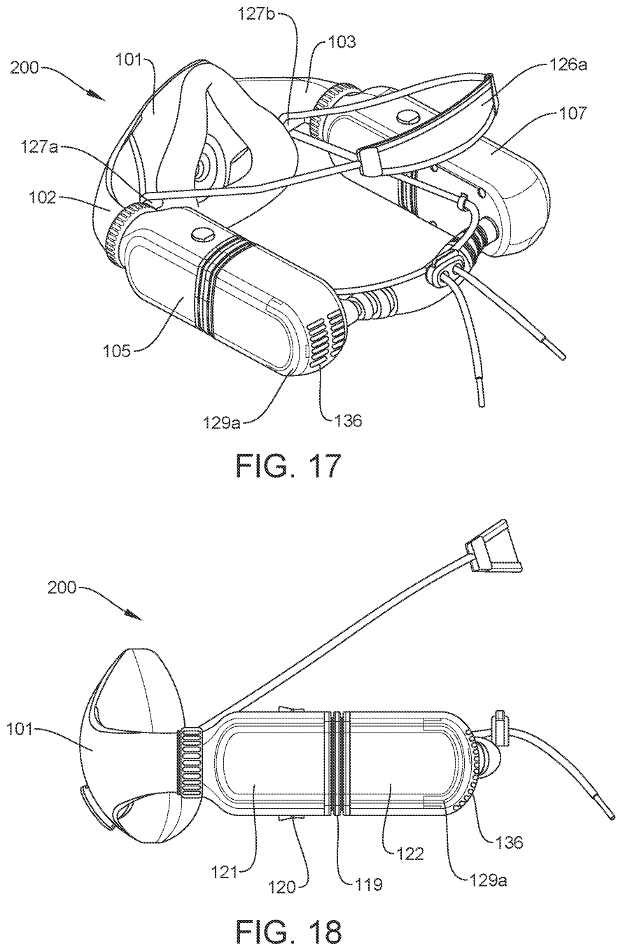

While the wearer's nose may support the portable respirator at the mask assembly 101 when worn, for improving the ergonomic aspect of the present invention, it may be preferable to wear the portable respirator with a head strap assembly 126 as shown in FIG. 10. The head strap assembly 126 will allow the wearer's head to provide support and decrease the weight or the load on a wearer's nose, when the portable respirator is worn. The mask assembly 101 may have head strap attachment means 127a, 127b or a bridge 127 positioned at the first end 102 and the second end 103 as shown in FIG. 17. Referring to an embodiment shown in FIGS. 10, 11, 12, 17, 27, 45, 46, the head strap assembly 126 may have a head loop portion and a neck loop portion. The head loop portion may be formed from a head band 126a with a first cord 126b extending to the first head strap attachment means 127a at a first end of the mask assembly, and a second cord 126c extending to the second head strap attachment means 127b the second end of the mask assembly. The head band 126a may be in connection to the first end of the first cord 126b and the first end of the second cord 126c. In another embodiment, the first cord 126b and second cord 126c form a continuous string and the head band 126a is a separatable strap where the string passes through. The neck loop portion may be formed from the first cord 126b and the second cord 126c extending from the respective head strap attachment means 127 to a toggle 126d engaging with the second end of the first cord and the second end of the second cord. For additional support, as shown in FIG. 37, the first housing 105 may have a first cord clip 126e mounted to the outer surface for securing a portion of the first cord 126b between the first head strap attachment means 127a and the toggle 126d; and similarly, the second housing 107 may have a second cord clip 126f mounted to the outer surface for securing a portion of the second cord 126c between the second head strap attachment means 127b and the toggle 126d.

In another preferred embodiment of a head strap assembly 126 is shown in FIG. 10. A head strap 126g may be used for engaging the head and a first side strap 126h may be received in the first head strap attachment means 127a, and a second side strap 126i may be received in the second head strap attachment means 127b. The head strap attachment means 127a, 127b may be a bridged structure such that the cord or straps may form a looped portion 127c, 127d with the engaging portion of the mask to a first end 102 or a second end 103 such that the cord or straps may be threaded through the looped portion 127c, 127d respectively. As the bridge structure 127a, 127b may be in consistent friction with the cord or strap of the head strap assembly, there may be a bridge sleeve 175 as shown in FIG. 47 for covering the bridged structure 127a, 127b. In a similar fashion to a toggle 126d, a strap hook 176 engaged with a strap loop 177 is in engagement with the second end of the first side strap 126h and the second side strap 126i. The movement of the strap 126h and 126i through the strap loop or strap hook tightens the neck loop portion of the head strap assembly. In one embodiment, there may be a head strap assembly that consists of two band pieces, one of which may be used to connect an elastic band at each side of the band piece. A hook and loop may be fitted to each of the elastic band respectively. Such a head strap may be fitted through the mask via its bridge structure, and the mask can be fitted to the face and fastened by the Hook 176 and Loop 177 mechanism independently before the rest of the portable respirator 100 are fitted to the head. Such a head strap assembly 126 may provide a simple way to don on or don off the respirator.

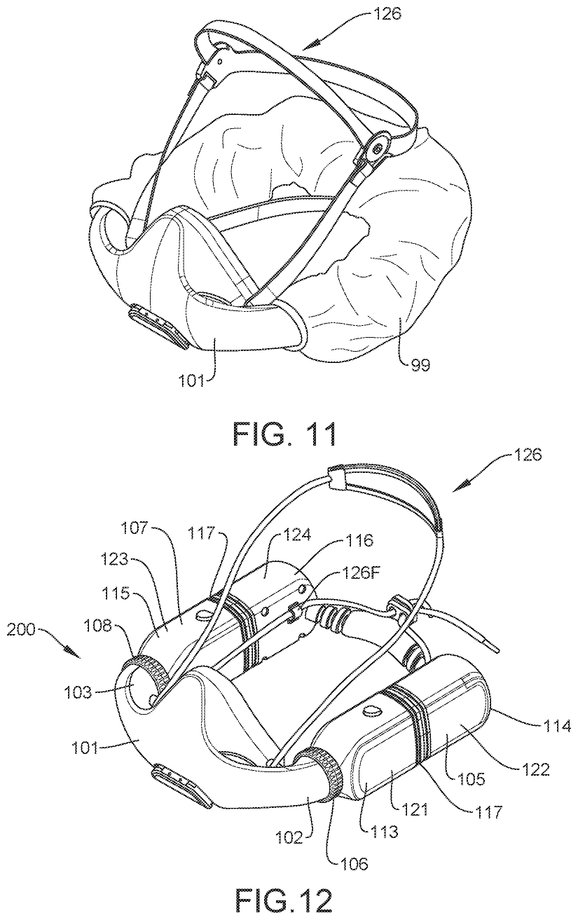

It may be appreciated that in one preferred embodiment, at least one equipment may be formed with the mask assembly 101. In another embodiment, the main unit 104 may be adapted for use in at least one specialised respiratory function selected from a group of: assisting user respiration, respiratory protection, breathing training, medical treatment, and breathing measurement and diagnostics. The generic modular respirator 100 may further comprise one or more sensors adapted to measure breathing related physiological parameters and the data measured and recorded by the equipment may be wireless communicated to a remote terminal. It may also be appreciated that the at least one equipment and/or sensors may be encapsulated in the mask assembly 101 or the main unit 104 in any order or configuration which allows the generic respirator to perform the at least one specialised respiratory function. To prevent dust collecting on the portable respirator, as shown in FIG. 11, the portable respirator may be covered by a dust cover 99.

Healthcare PAPR

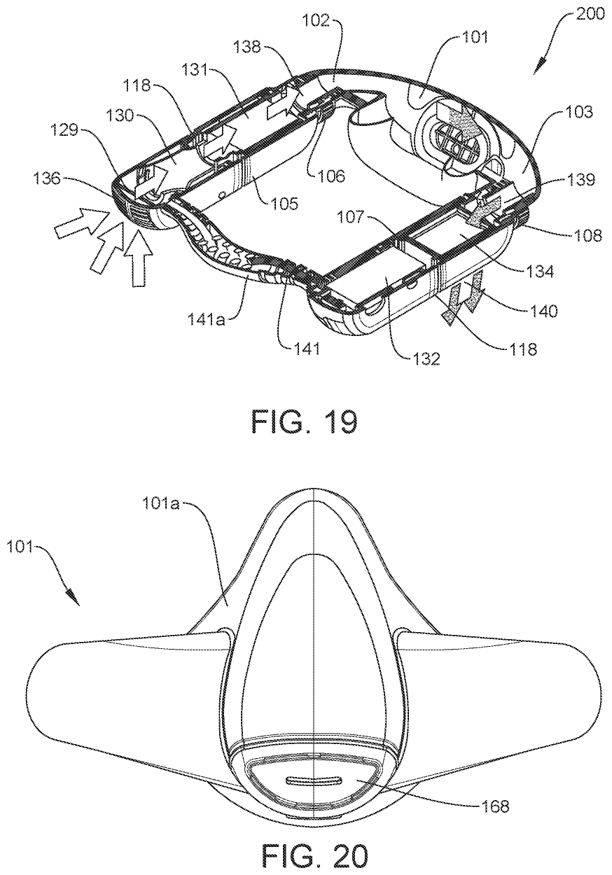

In one preferred embodiment of the generic modular respirator invention, as shown in FIGS. 12 to 19, the healthcare PAPR 200 for respiratory protection is adapted from the generic modular respirator 100 as shown in FIGS. 5 and 6. As illustrated in FIGS. 12, 17, 18, the head strap assembly 126 using the head band 126a is shown. It may be appreciated that the head strap assembly 126 using the head strap 126g may also be used as shown in FIG. 10. As shown in cross-sectional FIG. 19, the first housing 105 may encapsulate a prefilter 129, a blower 130 and a main filter 131. The second housing 107 may encapsulate a control unit 132 with a power source and an exhaust filter 134. The mask assembly 101 may be in fluid communication with the first housing 105 and the second housing 107. The blower 130 may be adapted for pumping the filter air from the first housing 105 to the mask assembly 101 via the first end of the mask assembly 102, wherein the first housing 105, the mask assembly 101 and the second housing 107 jointly form an air passage to pass therethrough. As there is an air outlet positioned in the main unit 104, the mask assembly 101 as shown in FIGS. 20 and 56, may not have an exhaust valve but may have a Bridge Piece that fits a speech diaphragm 167 and an inlet valve.

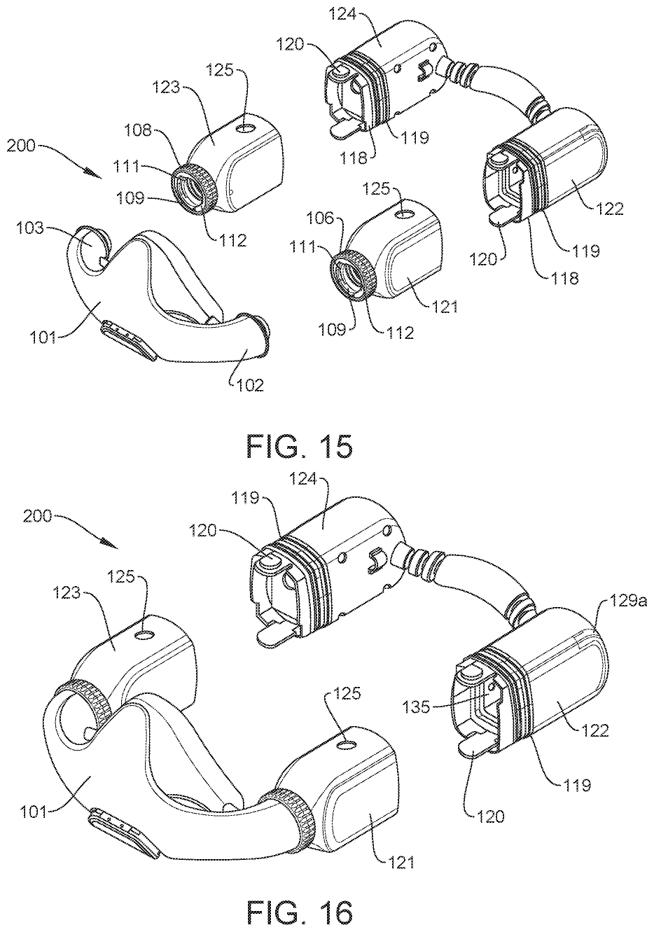

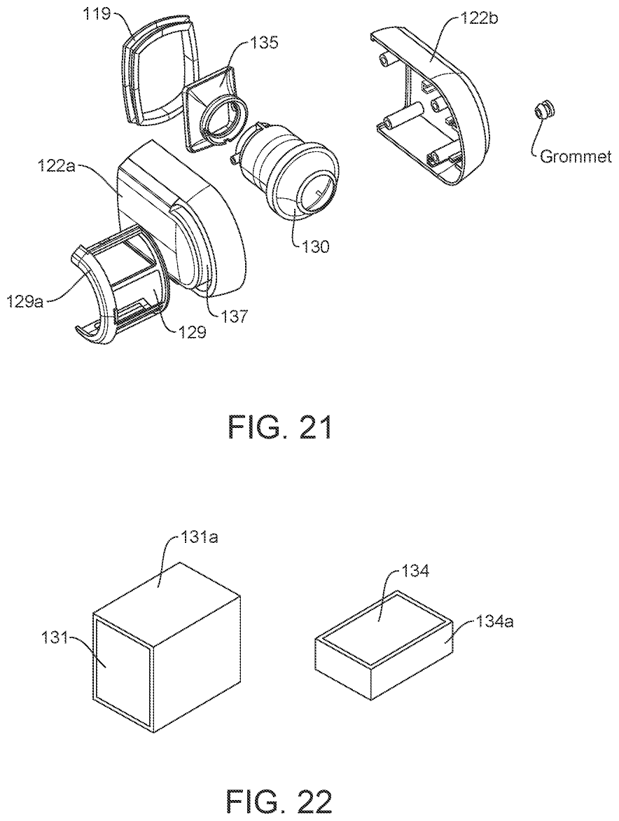

Preferably, the portable respirator 200 may use the housings 105/107 with the middle portion 117 with flexible concertina shaped walls. As shown in FIG. 15, 16, 19, 21, the Bellow-clip 118 of the first housing 105 may comprise an airflow guard or a bellow insert 135, wherein the blower 130 may be received or secured in the airflow guard or a bellow insert 135. The first distal portion of the first housing 122 may have a blower, a prefilter and an air inlet 136 for allowing the outside air to enter the first housing 105. After entering the air inlet 136, the external air is firstly filtered by a prefilter 129 as shown in FIG. 21. The prefilter 129 may be chosen from a washable or disposable fabric or paper product that may filter out particles greater than 3 microns, in which the prefilter 129 may filter out dust and/or allergens from the outside air before the blower 130 pumps the prefiltered air towards the main filter 131, in which the main filter 131 may be a HEPA filter that may filter out pathogens greater than 0.1 microns. An advantage of the prefilter 129 is that it protects and prolongs the filtering life of the main filter 131 by minimising the particles that required to be filtered. The prefilter 129 may be mounted or ultrasonically welded to a prefilter frame 129a, in which the prefilter frame 129a may be slotted in a prefilter slot 137 positioned after the air inlet 136, in which the prefilter 129 with the prefilter frame 129a is fully inserted into the prefilter slot 137, it seals the beginning of the air passage. Depending on the shape of the housings, as in this embodiment, there is a curved end at the first distal end 114 of the first housing 105, as shown in FIG. 21, the prefilter 129 and the prefilter frame 129a may be arcuate from an upper surface to the lower surface for better filtration as well as for better positioning between the air inlet 136 at the first distal portion 122 of the first housing 105 and the blower 130. An advantage of the arcuate prefilter 129 and the prefilter frame 129a is that the curvature extends the net area available for a prefilter media than otherwise in a flat shape. As also shown in FIG. 21, the first bellow-link 119 of the first Bellow-clip 118 may be adapted to receive a blower guard 135 and a blower 130 therethrough. The first distal portion 122 of the first housing 105 may have an outer shell half 122a and the other outer shell half 122b. There may be an over-molded seal positioned at the prefilter slot 137 in the outer shell half 122a and the other outer shell half 122b such that the beginning of the air passage is sealed when the prefilter slot 137 is slotted in it. As the blower 130 may generate noise in operation, especially when there the blower is near a user's ear when a user is wearing and using the portable respirator 200, it may be an advantage to provide a sound dampener surrounding the blower for user comfort and for protection of the person's ears. The sound dampener may be a sound absorbing foam, an acoustic foam, soundproofing insulation, cotton fibre or a fabric.

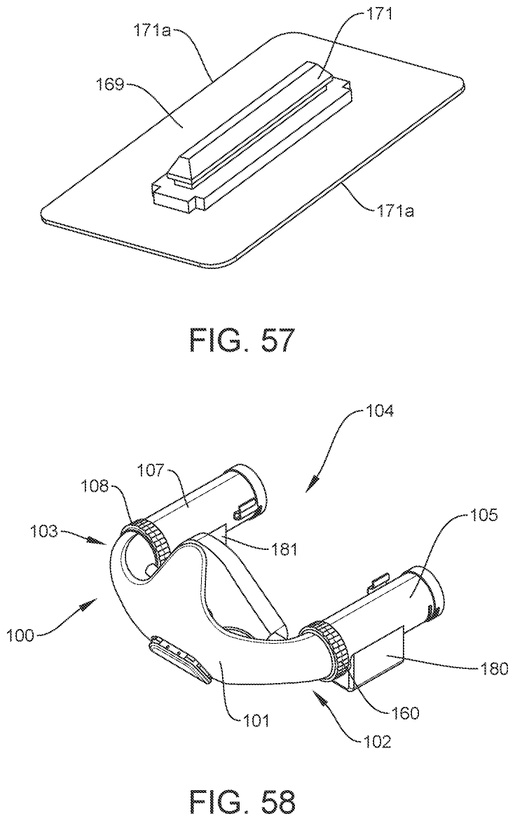

The blower 130 may pump the outside air through the prefilter 129, wherein the prefiltered air is pumped to the main filter 131, where the prefiltered air is HEPA filtered. As shown in FIG. 19, the HEPA filtered air then moves through a mask inlet 138 positioned at the first end 102 of the mask assembly 101. It may be appreciated that the mask inlet 138 may comprise a one-way valve such that exhaled air does not go back to the first housing 105. When the mask assembly is worn by the wearer, the filtered air and/or the exhaled air may exit the mask assembly 101 via the mask outlet 139 positioned at the second end 103 of the mask assembly 101. Once the filtered air and/or the exhaled air have exited the mask outlet 139 and into the second proximal portion 115 of the second housing 107, the air may be filtered by an exhaust filter 134 prior to exiting the second housing 107 via the air outlet 140 at the second proximal portion 123 of the second housing. It may be appreciated that the exhaust filter 134 may be similar to the main filter 131 in which the exhaled air is HEPA filtered so that any pathogens exhaled by the wearer will be filtered and not go back to the outside air for other people to breath in. The air outlet 140 may have an exhalation valve to ensure that the outside air does not go into the mask assembly 101 via the second housing 107. FIG. 57 illustrates one embodiment for the exhalation valve membrane 169, wherein, there is a long pivot 171 that allows the membrane 169 to open along the long sides 171a under positive pressure. As illustrated in FIG. 22, it may be appreciated that any one of the main filter 131 and the exhaust filter 134 may be adapted to be co-moulded or glued to a main filter frame 131a or an exhaust filter frame 134a respectively, wherein the filter frame may have a rectanguloid profile. The exhaust filter can be also a washable foam filter suitable to block droplets from the exhaled air with minimum flow resistance. The second distal portion 124 of the second housing 107 may encapsulate a control unit or an airflow control unit 132 with a power source for regulating the pumping strength of the blower 130 to maintain a positive air pressure for assisting the wearer to breathe. There is a sensing port 185a and 185b between the second proximal portion 123 and second distal portion 124 of the second housing as shown in FIG. 60, where an air-passing membrane may be fitted to block moisture and foreign substance to enter the sensing port. It may be appreciated that there may be at least one sensor, such as a flow sensor or a pressure sensor, which may be encapsulated by the main unit 104 such that the air flow and pressure may be measured. At least one sensor may be in communication with the control unit 132 such that the measured breathing parameter may influence the control unit 132 to dynamically regulate the pumping strength of the blower 130. The control unit 132 may be in wireless communication with the blower 130 or there may be an insulated electrical cable 141 in electrical connection between the air flow control unit 132 to the blower 130. With the embodiment using the insulated electrical cable, the first distal end of the first housing 114 and the second distal end of the second housing 116 may each have a cable outlet 142 such that one end of the insulated electrical cable may be received in the cable outlet 142 of the first housing 105, and the other end of the insulated electrical cable may be received in the cable outlet 142 of the second housing 107. For extra protection and for ergonomic support, the insulated electrical cable 142 may be covered by an elastic sleeve 141a. As shown in FIGS. 15 to 19, the sleeve 141a may also cover the cable outlets 142. As illustrated in FIG. 16, it may be appreciated that the detachable structures of the first and second housing via case-clip 120 allows easy access to the main filter and exhaust filter for replacement, and it also allows easy cleaning and sterilisation for the entire airpath comprising the mask assembly, the first and second proximal portions of the housings.

Healthcare APR

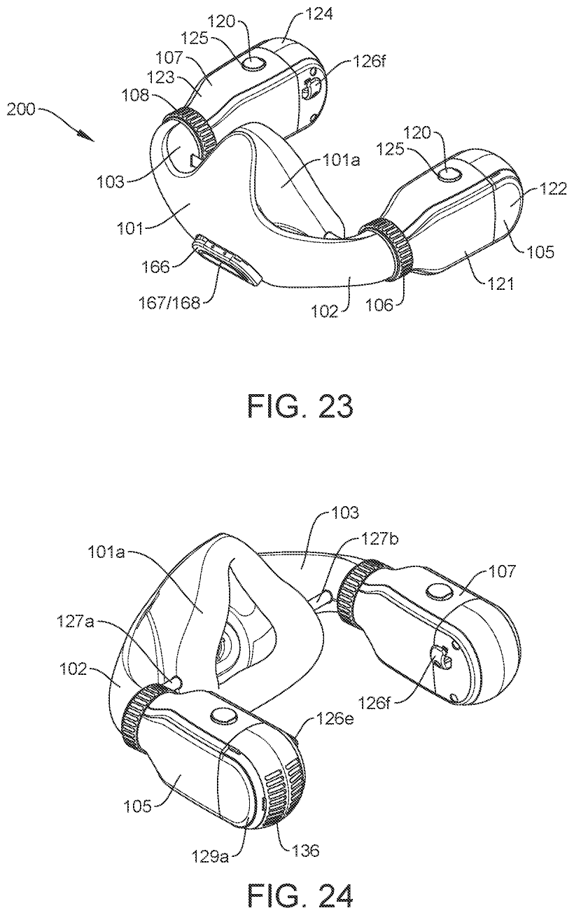

In another preferred embodiment of the generic modular respirator invention, as shown in FIGS. 23 to 26, the healthcare APR 200 for respiratory protection may have the same mask assembly 101, the proximal portion of the first housing 121 and the proximal portion of second housing 123 as in FIG. 12, as described in the Healthcare Respirator above. The first housing 105 may encapsulate a prefilter and a main filter. The second housing 107 may encapsulate an exhaust filter and an exhalation valve. The prefilter, the main filter, and the exhaust filter and the exhalation valve may be similar to the type described for the healthcare respirator. The mask assembly 101 may be in fluid communication with the first housing 105 and the second housing 107. This embodiment relies on the user's inhalation for moving the filter air from the first housing 105 to the mask assembly 101 via the first end of the mask assembly 102, wherein the first housing 105, the mask assembly 101 and the second housing 107 jointly form an air passage to pass therethrough. The first end of the mask assembly 102 may have an one way valve such that exhaled air does not enter the first housing but rather into the second housing 107 from the second end of the mask assembly 103. As there is an air outlet positioned in the main unit 104 at the second housing 107, the mask assembly 101 as shown in FIG. 20, may not have an exhaust valve but may have a speech diaphragm or a front mask cover 167/168. Similarly, the first housing 105 may have a first proximal portion 121 and a first distal portion 122, and similarly, the second housing 107 may have a second proximal portion 123 and a second distal portion 124. The first or second distal portion 122/124 may each have a case-clip 120 that may be adapted for connecting the proximal portion and the distal portion of the first housing or the second housing together, in which the proximal portion of the first housing or the second housing may each have a clip engagement means 125 for coupling the case-clip 120. As the air inlet is on the first distal portion of the first housing, the first distal portion and the first proximal portion of the first housing may jointly form an air passage to pass therethrough, wherein there may be a prefilter at the air inlet. As the air outlet is on the second proximal portion of the second housing, the second distal portion and the second proximal portion of the second housing may not jointly form an air passage to pass therethrough but with a blocked end. The first distal portion of the first housing 122 may have a first cord clip 126e, and the second distal portion of the second housing 124 may have a second cord clip 126f. The cord clips 126e, 126f may secure the cords of the headstrap. It can be obviously appreciated that the Bellow-clip and case-clip connection used in the generic respirator facilitate sharing of common module of mask assembly 101, the proximal portion of the first housing 121 and the proximal portion of second housing 123. Such a modular configuration allows easy interchangeability between an APR and PAPR.

Breathing Training Respirator

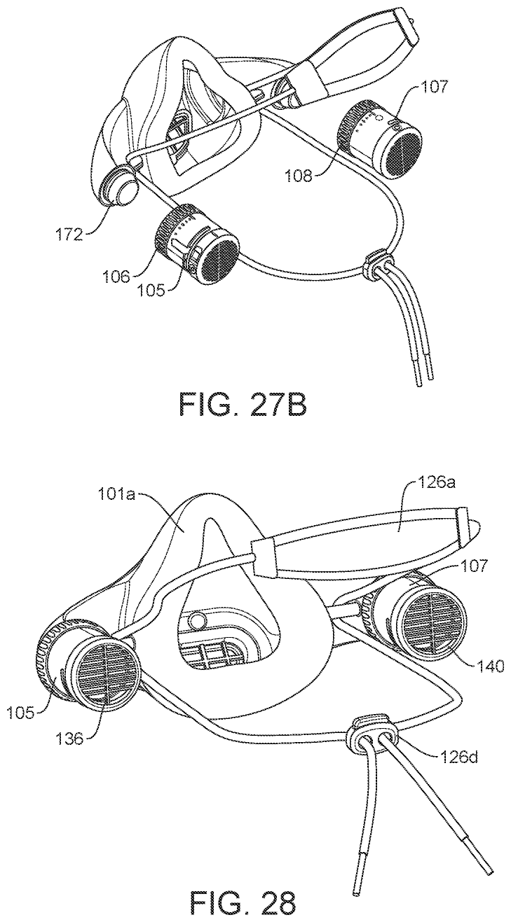

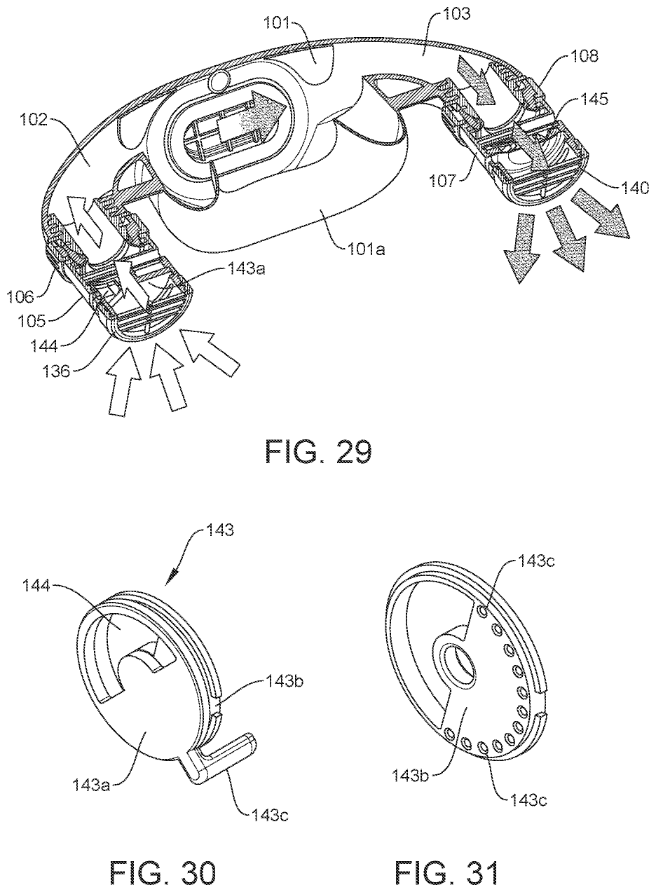

In another preferred embodiment of the generic modular respirator invention, as shown in FIGS. 27 to 36, the training respirator 300 for breathing training is adapted from the generic respirator 100 shown in FIG. 58 except without the Clip-on module. The first distal end of the first housing 114 may have an air flow dial 143 adapted to adjust a size of an opening 144 for outside air to enter the first housing 105 and for controlling inhalation resistance. The second distal end of the second housing 116 may have an exhaust membrane 145 for exhaled air to exit the second housing 107.

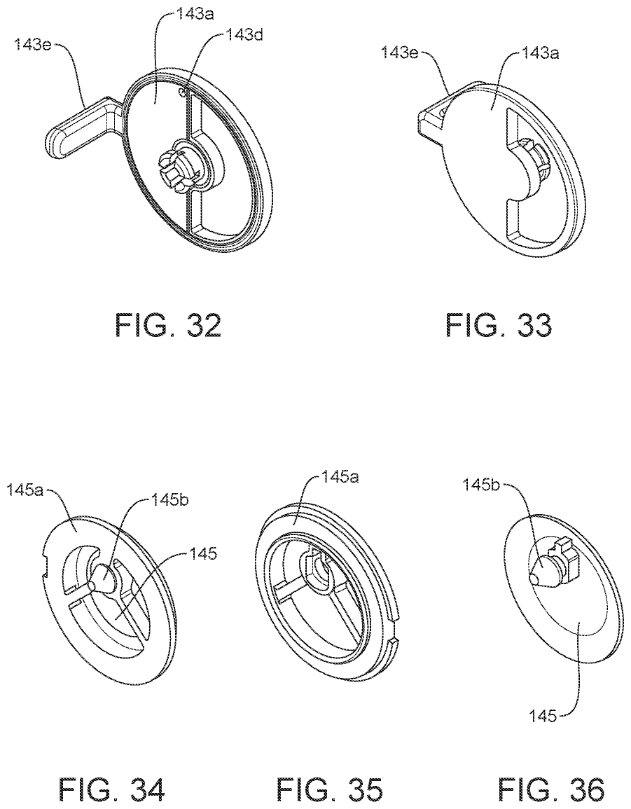

As shown in FIG. 30, the air flow dial 143 may comprise a multi-step setting to set the opening 144 into different predetermined sizes so that the wearer can select how much breathing resistance is used for their training session. The air flow dial 143 may comprise a first dial plate 143a (shown in FIGS. 32 and 33) and a second dial plate 143b (shown in FIG. 31). It may be appreciated that the two plates of the dial 143a, 143b will not cover all of the opening 144 when in use (shown in FIG. 30). The multi-step setting on the second dial plate 143b may be a series of concave semidomes 143c arranged near the curvature of the circumference of the second dial plate 143b. The concave semidomes 143c may be adapted to receive a protruding semidome 143d from the first dial plate 143a. There may be a dial handle 143e for manually rotating the first dial plate 143a from a first setting from one of the concave semidomes 143c to a next setting from an adjacent concave semidome 143c. While there may be twelve concave semidomes depicted in FIG. 31, it may be appreciated that there could be any number of concave semidomes 143c so that finer adjustment of the opening 144 can be achieved. When the air flow dial 143 is in its most opened position, the inhalation resistance is at a minimum.

As shown in FIGS. 34-36, there may be a circular exhaust membrane 145 (FIG. 36) pivotally connected to an exhaust membrane frame 145a (FIG. 35). The pivotal connection may be a pivot pin 145b which may be off-centre from the exhaust membrane 145 thus the membrane 145 works as a flap which can flap away from the exhaust membrane frame 145a such that the air flow is directed towards the far end of the pivot. The advantage of the off-centre pivoted membrane is to achieve a lower flow resistance compared with a centre pivoted membrane of the same diameter, especially when the membrane has to be small.

It can be obviously appreciated that mask assembly 101 and the Push-fit & Twist-release connection as described in the generic respirator 100 are adapted here,

Breathing Training Respirator with Electronic Modules

Similar to some of the components used for the breathing training respirator, such as an airflow dial in the first housing, and the exhalation valve in the second housing, as shown in 37 to 39, 58, 59a, 59b, the first housing and second housing of the portable respirator 300 may each further comprise a flow element 182/183 and an electronic module 180/181, wherein the flow element in the first housing may produce signals for the inhaled flow, and the flow element in the second housing may produce signals for the exhaled flow. In one preferred embodiment, each of the electronic modules may have a Clip-on mechanism as shown in FIGS. 59a and 59b, wherein the electronic modules can be retained to the first and second housings that can be easily removed by a user for cleaning or servicing. In one preferred embodiment, each of the distal end of the first and second housing may have a fixed add-on module as shown in FIGS. 59a and 59b, wherein the add-on module in the first hosing may comprise an air flow dial, wherein the add-on module in the second hosing may comprise an exhaust valve. In another preferred embodiment, each of the distal end of the first and second housing may have a releasable add-on module with push-fit and twist-release connection as shown in FIGS. 59c and 59d, wherein the add-on module with push-fit and twist-release connection in the first hosing may comprise an air flow dial, wherein the add-on module with push-fit and twist-release connection in the second hosing may comprise an exhaust valve. It is obviously appreciated that the add-on module with push-fit and twist-release connection in the first and second housing is identical to the first and second housing as shown in FIG. 27b. Such a configuration allows interchangeability between training respirators with and without electronics modules. The electronics modules may comprise components, such as one or more flow sensors, one or more pressure sensors and an electronic actuator (not shown) which may dynamically control the air flow dial 143 or the breathing resistance based on the measured breathing of the wearer for an electrically controlled programmable resistance breathing training. The electronic actuator may be one selected from a group of: a solenoid valve, and a blower for blowing against the direction of inhalation. The electronic actuator may be fitted in a fixed add-on module at the distal end of the first housing, wherein an electrical cable is passed and connected between the electronics module and the electronics actuator. The electronics modules may have wireless communication means to communicate with a remote device, such as a mobile phone, a smart watch or any suitable devices with cooperating wireless communication capacity. The electronic modules may measure breathing effort for a breathing training. Preferably, the breathing effort may be derived based on the flowrate and pressure from breathing, such that, the power of the breathing effort is the product of the breathing flow and pressure, the work of the breathing effort is the product of tidal volume and pressure. By measuring breathing power and work, the resistance loading to the breathing muscles can be quantified, and on the other hand, breathing power and work may be used to quantify for breathing efficiency for a given exercise, thus it would help improve the breathing training over the currently available training masks. The electronic modules may also provide other breathing physiology measurement, such as breath rate, tidal volume and minute ventilation, for breathing efficiency training, breathing normalisation training as well as general breathing health monitoring.

Breathing Measurement Apparatus

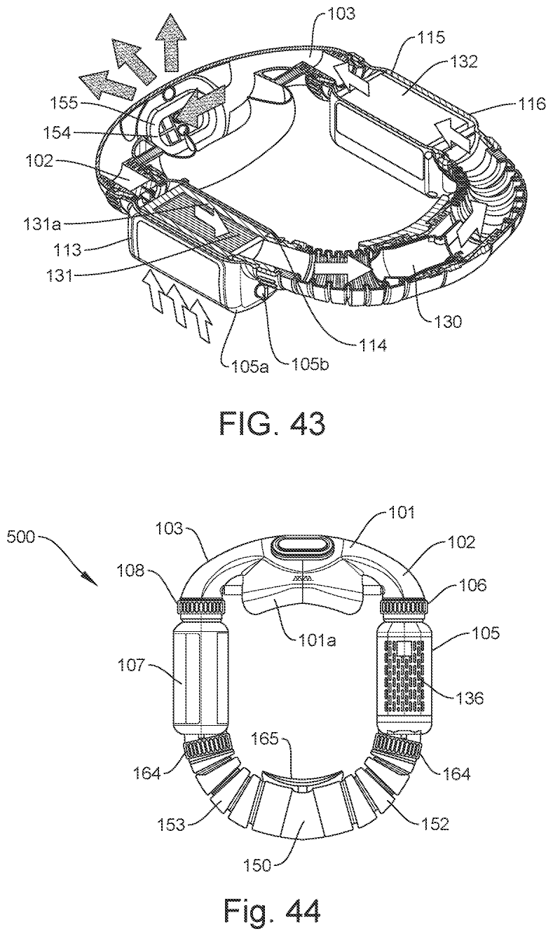

In another preferred embodiment of the generic respirator invention, as shown in FIG. 40, the measurement respirator 400 is to provide an advanced breathing measurement and a tool to normalise persons breathing. The respirator is adapted from the generic respirator 100 with a neck assembly 128 as shown in FIG. 4. The first housing 105 may encapsulate a battery or a power source and a desiccator 146, and the second housing 107 may encapsulate a control unit or an airpath control unit 132 and a breathing airway. The mask assembly 101 may encapsulate a breath sampler 147 and the neck assembly 128 may be a flexible concertina shaped neck assembly 128 which may encapsulate a pump 148 and a CO₂ sensor 149. The pump 148 may be adapted for pumping sampled air received in the breath sampler 147 to the CO₂ sensor 149 via the desiccator 146. In one embodiment, the pump 148 is the same as the blower 130 as shown in FIG. 43. The desiccator 146 may contain a desiccant for removing the moisture of the exhaled sampled air prior to being sensed by the CO₂ sensor 149 as moisture may affect the reading of the CO₂ sensor 149. There may be a tube 147a in connection between the breath sampler and the desiccator 146, and there may be another tube 147b in connection between the desiccator 146 and the CO₂ sensor 149.

The first housing 105 may be modular such that the desiccator 146 and/or the desiccant may be easily assessable for replacement when the desiccant may have absorbed a predetermined amount of moisture from a period of use. The neck assembly 128 may be covered by a bellow, wherein an electrical cable runs between the CO2 sensor 149, the pump 148, and the control unit 132, wherein the control unit control the pump and collect CO2 sensor signal via the electrical cable, wherein the CO2 sensor, the pump connect to the desiccator 146 via an internal tube, wherein the bellow may connect to the first distal end of the first housing 114 and the second distal end of the second housing 116 via thread connectors. The neck assembly 128 may have an air outlet at the middle section of the 150 (not shown) for sampled air to be pumped outside the main unit 104. The bellow may have a disengageable middle section 150 at the juncture that may encapsulate a pump 148 and/or the CO₂ sensor 149 such that the pump 148 and/or the CO₂ sensor 149 may be easily accessed for allowing service to access the equipment.

Preferably, the second housing 107 comprises a single airway 140 for air to enter and to exit from the respirator 400, as well as facilitating respiratory flow and pressure measurement.

Industrial Respirator

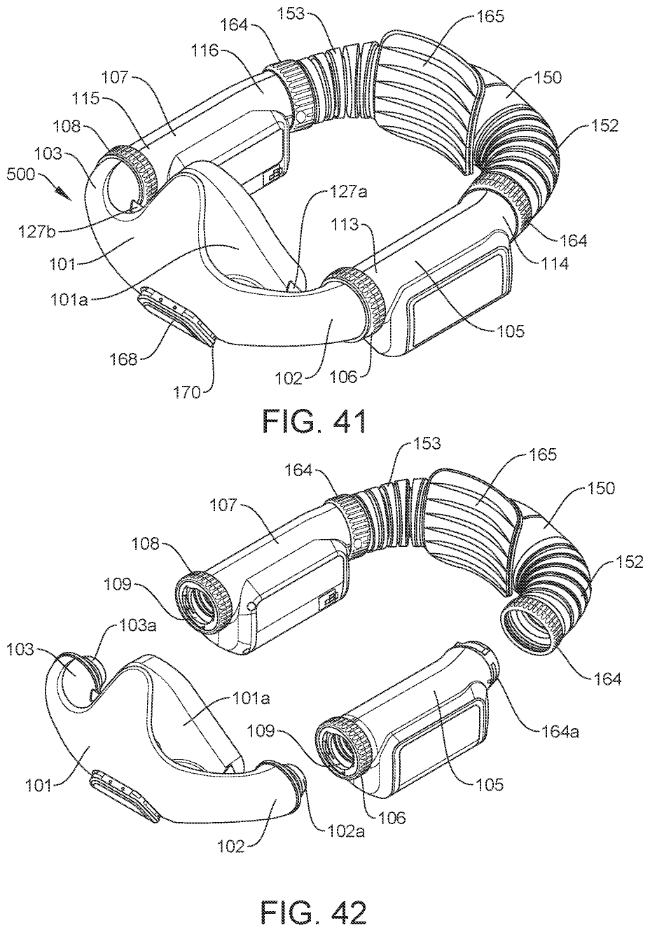

In another preferred embodiment of the invention, as shown in FIGS. 41 to 55, the industrial respirator 500 for filtering particulates in industrial occupations is adapted from the generic modular respirator 100 with a neck assembly 128 as shown in FIG. 4. The first housing 105 may encapsulate a prefilter 129 and a main filter 131. The second housing 107 may encapsulate a control unit 132, wherein there may be a battery 163a, a main control PCB or controller 162, a sealed airpath 158. The neck assembly 128 which may be a bellow and the neck assembly 128 may encapsulate a blower 130 at the middle section 150. The blower 130 may be adapted for pumping the filtered air from the first distal end of the first housing 114 to the mask assembly 101 via the neck assembly 128 and the second distal end of the second housing 116, such that as shown in FIG. 43, ambient air is drawn in from the bottom of the first housing 105, filtered by the prefilter and the main filter, entering the neck assembly at the distal end of the first housing, passing the blower before exiting the neck assembly at the distal end of the second housing, then enter the control unit via the sealed airpath before entering the mask at the proximal end of the second housing.

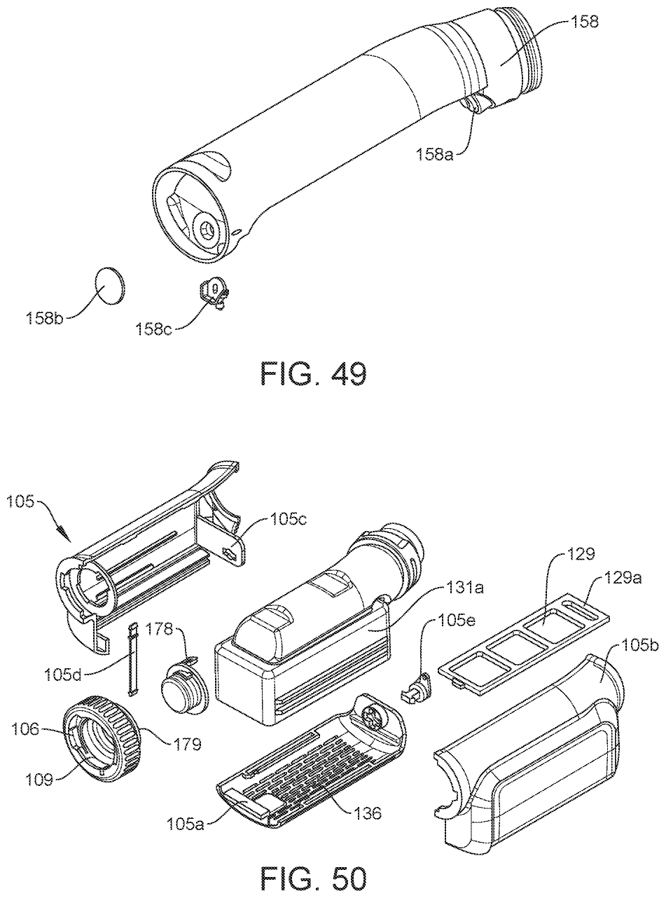

The components of the second housing 107 are shown in FIG. 48 and the sealed airpath 158 are shown in FIG. 49. The controller 162 may be in electrical communication via an insulated electrical cable 141 with the blower 130, wherein the main control PCB or controller 162 measures the pressure in the main unit 104, and based on the pressure measurement, the controller 162 may have electronics that allows for dynamically regulating the blowing strength of the blower 130 such that a positive pressure in the mask assembly is maintained.

The middle section 150 may be disengageable with a first bellow portion 152 and a second bellow portion 153 of the neck assembly 128. The first bellowed portion 152 may be in connection with the first distal end of the first housing 114 with a releasable bayonet connector 164a and the second bellowed portion 153 may be in connection with the second distal end of the second housing 116 with a fixed connector 164. The front of the mask assembly 101 shown in FIG. 47 may have an oval shaped exhalation valve membrane 169 for exhaled air to exit the portable respirator 500, wherein the membrane 169 has two pivot pins to secure the membrane to the seat 170 and to allow the membrane to open along the two shorter ends under positive pressure. The exhalation valve membrane 169 may be further secured by an exhalation valve cover 168.

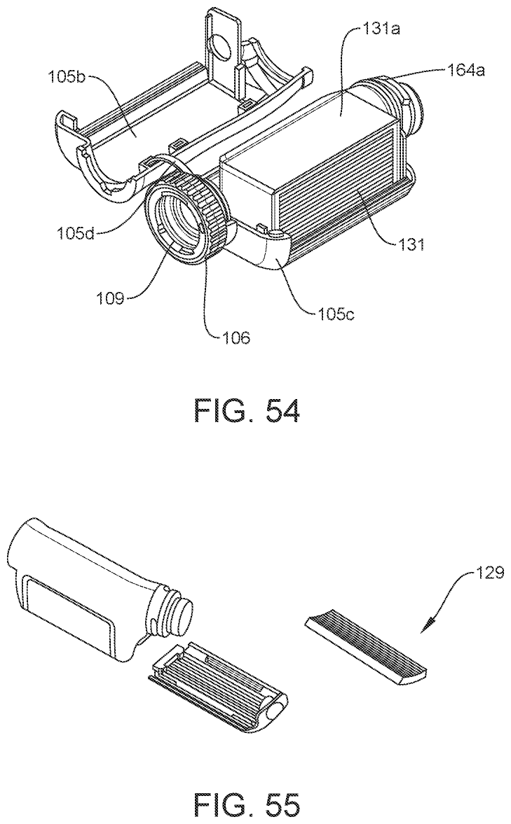

As shown in FIGS. 50 to 54, the first housing 105 may have an air inlet 136 at the filter door 105a of the first housing for receiving air from the outside environment transversely to the airflow direction. The filter door 105a may be detached from the first housing 105, in which the locking mechanism 105e can secure the filter door to the first housing. The filter door is shown to contain an airlet 136. The first housing 105 may have a shell half 105b in connection with the other shell half 105c, in which a tether 105d is in connection with the left outer shell 105b and the right outer shell 105c so that the shells 105b, 105c are not far from each other when the first housing 105 is disassembled, to minimise losing parts, which is also shown in FIG. 54. The main filter may have an integrated airpath between the main filter frame 131a and the air outlet 164a of the main filter, wherein filter outlet 164a is also the outlet of the first housing 105, such that the main filter ensures leak-free connection to the neck assembly. There may be a prefilter frame 129a with a prefilter 129 between the air inlet 136 and the base of the main filter 131. As the airpath for this embodiment does not go from the first proximal end of the first housing 113 to the first end of the mask assembly 102, there may be plug 178 for plugging the other end 179 of the first circular Push-fit socket 109.

The prefilter 129 and the main filter 131 may be the same type as used in the embodiment of the healthcare respirator. In another embodiment, prefilter 129 may be a curved pleated element as shown in FIG. 55. Due to the compact design of the first housing 105, the cross-section of the air inlet 136 may be limited, a pleated prefilter helps increase the effective flow-pass area thus reducing flow resistance.

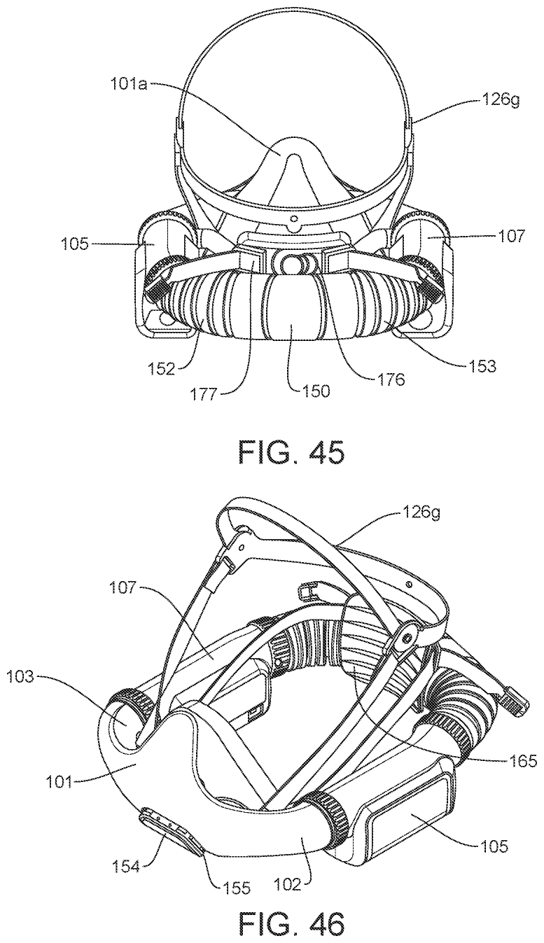

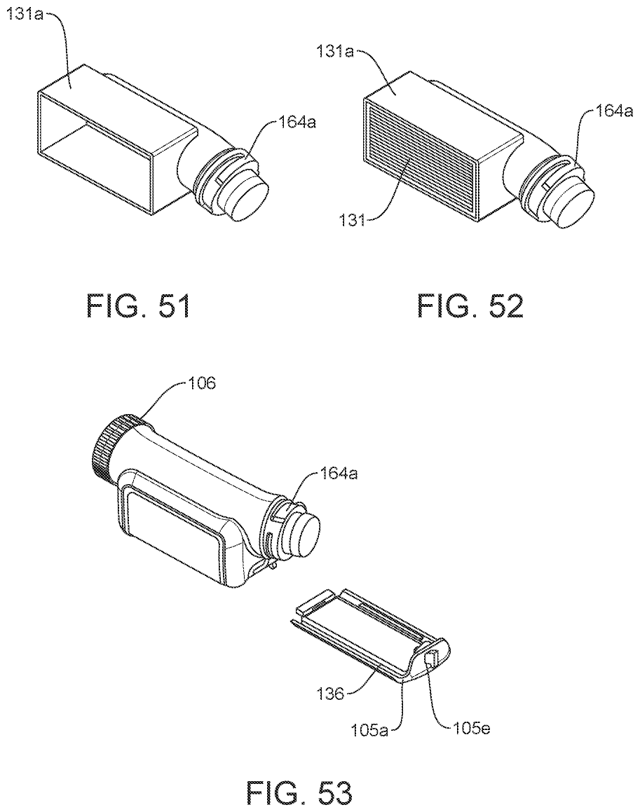

As shown in FIG. 48, the second housing 107 may have an inner case on one side 156, and the other side 157 in which the case covers or encapsulates an airpath unit 158. As shown in FIG. 49, the sealed airpath unit 158 may comprise a grommet 158a to seal the entry of the electrical cable linking between the blower and the controller, a vent membrane 158b for protecting the pressure port from moisture, and a pressure port 158c. There may be a vent cover membrane 158d for covering the vent membrane (not shown) at the bottom of the inner case 156. There may be a door for a DC Jack 159 mounted to the inner case 156 and a lens control panel 160 mounted to the other side of the inner case 157 which may also encapsulate a rubber keypad 161 and keypad PCB 162a and a main printed circuit board or controller 162. It may be appreciated that a supporting tray 163 and the main printed circuit board 162 form a potting main PCB for reliability and safety. The second distal end of the second housing 116 may be connected to the second bellow portion 153 of the neck assembly 128 via a screw nut connector 164. Similarly, the first distal end of the first housing may be connected to the first bellow portion 152 of the neck assembly 128 via another screw nut connector 164. It may be appreciated that other types of connection may be suitable. For example, as shown in FIGS. 51 and 52, the distal ends of the first and second housing may have a bayonet type connector 164a. Similarly, the middle section 150 of the neck assembly may be a hard piece which is disengageable at the juncture for allowing service to access the blower 130. As the middle section 150 is a hard piece, there may be a soft neckpad 165 mounted to the middle section 150 such that the neck of the wearer can rest on the neckpad 165 when in use. As shown in FIGS. 45 and 46, the neckpad 165 mounted to the middle section 150 may define a buckle/toggle retaining means such that when using together with the head strap assembly 126, the buckle or the toggle 176/177 may be received at the retaining means so that the buckle or the toggle is in contact with the back of the neckpad 165, so that the head strap pulls the neck assembly against back of the neck to secure the respirator around the head, and so that not in contact with the wearer's neck for comfort.

It can be obviously appreciated that the Breathing Measurement Apparatus and the Industrial Respirator described above are in similar housing and neck assembly configuration but with different internal parts performing different functions. In a more general term, the generic respirator invention is not restricted to a particular functional respirator, such as for protection purpose. The invention provides a respirator that can be configured or adapted to any applicable functional respirators. Therefore, apart from the novel connections in linking the modular parts within the generic respirators, the main unit can be configured by any variations of the described housings with or without the neck assembly, with or without the middle section in each housing, with or without bellow-clips, bellow-links, case-clips, clip-on connectors, bayonet or thread connectors, push-fit and twist-release connectors, wherein they can contain any parts, in any sequences as applicable to any particular breathing function a target respirator intended to achieve. Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1. A generic modular respirator comprising:
a mask assembly having a first end and a second end;
a replaceable main unit having a first housing with a first push-fit and twist-release connector to engage the first end of the mask, and a second housing with a second push-fit and twist-release connector to engage the second end of the mask; wherein the mask assembly and the main unit are adapted to perform breathing functions, such as breathing protection, breathing training, breathing measurement and diagnostics;
such that the mask assembly is adapted to be quickly fitted to and released from the main unit by actuating the first and second push-fit and twist-release connectors.

2. The generic modular respirator of claim 1, wherein the first push-fit and twist-release connector comprises a first circular air-passing socket and a first rotatable control ring with a latching spring, wherein the second push-fit and twist-release connector comprises a second circular air-passing socket and a second rotatable control ring with a latching spring;
wherein the first end of the mask assembly has a first pairing air-passing stud to connect with the first push-fit and twist-release connector of the main unit and wherein the second end of the mask assembly has a second pairing air-passing stud to connect with the second push-fit and twist-release connector of the main unit;
wherein the first and second air-passing studs can each freely rotate within the first and second Push-fit & Twist-release connectors respectively.

3. The generic modular respirator of claim 1 wherein the mask assembly has an internal bridge piece, wherein the mask assembly comprises one or more inlet valves, a speech diaphragm or an exhalation valve, wherein the bridge piece stiffens the internal cavity of the mask.

4. The generic modular respirator of claim 1 wherein the first housing and the second housing each comprises a middle portion with flexible concertina shaped walls;
wherein the middle portion is a connector assembly comprises a bellow-link and a case-clip, wherein the connector assembly is each adapted for connecting the proximal portion to the distal portion of the housings, such that the bellow-link allows the housings to arcuate when the neck abuts the middle portion, such that the case-clip allows modularity between the proximal portion of the housing and the distal portion of the housing, such that the case-clip allows servicing the proximal portion and the distal portion of the housing differently.

5. The generic modular respirator of claim 1 wherein the first housing and the second housing each comprises a distal portion with a case-clip.

6. The generic modular respirator of claim 1, wherein the first housing and the second housing each comprises a Clip-on module at the bottom of the housing, wherein the Clip-on module can be removed from the first housing and the second housing, wherein the first housing and second housing can be washed and serviced, wherein the first housing and the second housing each comprises an add-on module with or without a push-fit and twist-release connector at the distal portion of the housing.

7. The generic modular respirator of claim 1 wherein the first housing and the second housing are further linked by a neck assembly having a first end and a second end, wherein the first end of the neck assembly is adapted to engage with a first distal end of the first housing, and the second end of the neck assembly is adapted to engage with a second distal end of the second housing, wherein the neck assembly provides a channel for fluid communication and/or for electrical communication between the first housing and the second housing;
wherein one or more connections between the neck assembly and the housings are fixed;
wherein one of more connections between the neck assembly and the housings are removeable by a bayonet or thread connectors.

8. The generic modular respirator of claim 1 wherein the mask assembly has a head strap assembly attachment means for securing a head strap assembly to the mask assembly;
wherein the first housing, the second housing and the neck assembly have cooperating means to engage with the head strap assembly for securing the respirator to the head.

9. The generic modular respirator of claim 4, wherein the respirator is adapted as Healthcare PAPR to be used for respiratory protection; wherein the first housing encapsulates a prefilter, a blower and a main filter; wherein the second housing encapsulates a control unit with a power source, an exhalation valve and filter;
wherein the mask is in fluid communication with the first housing and the second housing;
wherein the blower is adapted for pumping the filtered air from the first housing to the mask, wherein the exhaled air exits from the mask to the second housing, wherein the first housing, the mask and the second housing jointly form an air passage to pass therethrough.

10. The generic modular respirator of claim 9, wherein the connector assembly of the first housing comprises an airflow guard, wherein the blower is received in the airflow guard;
wherein the first distal portion of the first housing has an air inlet, and wherein a first proximal portion of the first housing has an air outlet;
wherein the second proximal portion of the second housing has an exhaust inlet, an exhaust outlet; such that the exhalation valve and filter are positioned between the exhaust inlet and the exhaust outlet;
wherein the exhalation valve membrane has a long pivot that allows the membrane to open along the long sides under positive pressure;
wherein there is a pressure sensing port between the proximal and distal portion of the second housing, wherein the sensing port uses an air-passing membrane to block moisture and foreign substance to enter the sensing port;
wherein by removing the distal portions of the first and second housing, both the main filter and exhaust filter can be replaced, and the entire airpath comprising the mask assembly, the proximal portion of the first and second housings can be washed or sterilized.

11. The generic modular respirator of claim 9, wherein the first distal portion has a prefilter slot adapted for receiving a prefilter frame, wherein the prefilter is mountable to the prefilter frame; and wherein a cross-sectional shape of the slot is arcuate from an upper surface to a lower surface of the first distal end;
wherein any one of the main filter and the exhaust filter is adapted to be co-moulded or glued to a filter frame, wherein the filter frame has a rectanguloid profile;
wherein the exhaust filter is optionally a washable foam filter.

12. The generic modular respirator of claim 9, wherein the first distal end of the first housing and the second distal end of the second housing each has a cable outlet for receiving insulated electrical cables such that the control unit is in electrical communication with the blower;
wherein the insulated electrical cable is covered by an elastic sleeve.

13. The generic modular respirator of claim 5, wherein the respirator is adapted as a healthcare APR to be used for respiratory protection; wherein the mask assembly, the first proximal portion of the first housing and the second proximal portion of the second housing having a Healthcare PAPR structure; wherein the first housing encapsulates a main filter; wherein the second housing encapsulates an exhalation valve and filter;
wherein the mask is in fluid communication with the first housing and the second housing;
wherein the first distal portion of the first housing has an air inlet, and wherein a first proximal portion of the first housing has an air outlet;
wherein the second proximal portion of the second housing has an exhaust inlet, an exhaust outlet; such that the exhalation valve and filter are positioned between the exhaust inlet and the exhaust outlet;
wherein the exhalation valve membrane has a long pivot that allows the membrane to open along the long sides under positive pressure;
wherein the distal portion of first housing has a case-clip, a cord clip, a prefilter and an air inlet;
wherein the distal portion of the second housing has a case-clip, a cord clip and a blocked end;
wherein by removing the distal portions of the first and second housing, both the main filter and exhaust filter can be replaced, and the entire airpath comprising the mask assembly, the proximal portion of the first and second housings can be washed or sterilized;
wherein the healthcare APR is interchangeable.

14. The generic modular respirator of claim 7 wherein the respirator is adapted to be used as an Industrial PAPR for filtering particulates or gas in industrial occupations;
wherein the front of the mask assembly comprises an exhalation valve; wherein the first housing encapsulates a prefilter, and a main filter;
wherein the second housing encapsulates a controller, a battery pack and a sealed airpath;
wherein the neck assembly encapsulates a blower, wherein the blower is adapted for pumping the filtered air from the first distal end of the first housing to the mask assembly via the second distal end of the second housing;
wherein the neck assembly comprises a bellow connector engageable between a first bellowed portion and a second bellowed portion, wherein the first bellowed portion is in connection with the first distal end of the first housing with a releasable bayonet connector, and the second bellowed portion is in connection with the second distal end of the second housing with a fixed connector; wherein the bellow connector is adapted to receive a blower therein;
wherein the controller is in electrical communication with the blower; wherein the controller measures the pressure in the mask, and based on the pressure measurement, the controller dynamically regulates the blowing strength of the blower such that a positive air pressure in the mask assembly is maintained.

15. The generic modular respirator of claim 14, wherein the first housing comprises two shell halves linked by a tether;
wherein the two shell halves are closed by a filter door and secured by a lock; wherein the main filter comprises a pleated paper section at the air inlet, and an integrated airpath at the air outlet;
wherein the integrated airpath of the main filter has a bayonet connection at the outlet for releasably connecting with the neck assembly;
wherein the first circular push-fit socket is blocked by a plug; wherein a curved pleated prefilter is fitted with the filter door;
wherein the exhalation valve membrane is in oval shape with two pivot pins, wherein the membrane opens along the short ends under positive pressure.

16. The generic modular respirator of claim 14 wherein the sealed airpath in the second housing transports filtered air from the distal end to the proximal end;
wherein an electrical cable is entered at the distal end of the airpath to link between the blower and the controller, wherein the cable entry is sealed by a grommet;
wherein the middle section of the neck assembly has a soft neckpad, wherein there is a retaining means to help engage with the head strap buckle, wherein the head strap pulls the neck assembly against back of the neck to secure the respirator around the head.

17. The generic modular respirator of claim 7 wherein the respirator is adapted to be used as a measurement respirator;
wherein the first housing encapsulates a battery and a desiccator, and wherein the second housing encapsulates a control unit and a breathing airway;
wherein the mask encapsulating a breath sampler;
wherein a flexible concertina shaped neck assembly encapsulating a pump and a CO₂ sensor; wherein the pump is adapted for pumping sampled air from the breath sampler to the CO₂ sensor via the desiccator;
wherein the control unit is in electrical communication with the pump and the CO₂ sensor; wherein the control unit measures and records the breathing related physiological data, and wherein the recorded data is wirelessly communicated from the control unit to a remote terminal unit;
wherein the desiccator is in fluid communication between the breath sampler, and the CO₂ sensor;
wherein the measurement respirator provides a tool to normalise persons breathing.

18. The generic modular respirator of claim 6 wherein the respirator is adapted to be used as a training respirator;
wherein a first distal end of the first housing has an air flow dial adapted to adjust a size of an opening for air to enter the first housing, and for controlling inhalation resistance;
wherein the air flow dial comprises a multi-step setting to set the opening into different predetermined sizes;
wherein a second distal end of the second housing has a circular exhaust membrane for exhaled air to exit the second housing;
wherein the circular exhalation valve membrane has an off-centre pivot pin.

19. The generic modular respirator of claim 6, wherein the respirator is adapted to be used as a training respirator with breathing measurement;
wherein the first and second housing each has a Clip-on module;
wherein the first housing has a flow element to produce signals for the entered airflow; wherein the second housing has a flow element to produce signals for the exhaled flow;
wherein the distal portion of the first and second housing each has an add-on module with or without a push-fit and twist-release connector;
wherein the add-on module of the first housing has an air flow dial adapted to adjust a size of an opening for air to enter the first housing;
wherein the add-on module of the second housing has a circular exhaust membrane for exhaled air to exit the second housing;
wherein the circular exhalation valve membrane has an off-centre pivot pin; wherein the clip-on modules on the first and second housings each comprises a sensing PCB and a battery;
wherein the clip-on modules sense the signals for the entered and exhaled air for measuring breathing data;
wherein the breathing data include power of breathing, work of breathing, and other breathing data;
wherein power of breathing and work of breathing data are used to quantity the breathing effort of the user against the resistance imposed by the respirator;
wherein power of breathing and work of breathing are used to quantity the breathing efficiency of the user when doing certain exercises;
wherein the sensing PCBs transmit the measured breathing data to remote devices.

20. The generic modular respirator of claim 6, wherein the respirator is adapted to be used as a training respirator with breathing measurement and programmable breathing resistance;
wherein the add-on module of the first housing comprises an electronic actuator to control the air flow dial or breathing resistance based on the measured breathing related physiological parameters;
wherein the add-on module is in fixed connection to the first housing to allow electrical connection between the first housing and the add-on module;
wherein the electronic actuator is one selected from a group of: a solenoid valve, and a blower for blowing against the direction of inhalation;
wherein the breathing resistance is electrically controlled and is programmable.

21. The generic modular respirator of claim 1:
wherein the mask assembly covers nose, or nose and mouth, or nose and mouth and eyes;
wherein the mask assembly fits no or more speech diaphragms, no or more inlet valves, no or more exhalation valves;
wherein the main unit can be adapted to a specific functional respirator with or without bellow-clips, bellow-links, case-clips, clip-on connectors, bayonet or thread connectors, push-fit and twist-release connectors;
wherein the main unit contains any parts, in any sequences to perform any breathing functions.
